(19) United States
(12) Patent Application Publication
(10) Pub. No.: US 2012/0156202 A1
(43) Pub. Date: Jun. 21, 2012

(54) AGE RELATED MACULAR DEGENERATION TREATMENT

(76) Inventors: Totada R. Shantha, Stone Mountain, GA (US); Jessica Shantha, Stone Mountain, GA (US); Erica Maya Shantha, Stone Mountain, GA (US)

(21) Appl. No.: 13/407,681

(22) Filed: Feb. 28, 2012

Related U.S. Application Data
(63) Continuation-in-part of application No. 12/940,247, filed on Nov. 5, 2010.

(51) Int. Cl. A61K 38/28; A61P 27/02; A61P 9/00; A61K 39/395

(52) U.S. Cl. 424/134.1; 514/5.9; 424/141.1; 424/133.1; 514/6.5

(57) ABSTRACT

A method for treating age related macular degeneration (AMD) using an insulin preparation applied topically to the conjunctival sac of the affected eye. Another aspect of this invention is using antiangiogenic adjuvant therapeutic agents such as bevacizumab, ranibizumab, pegaptanib, etanercept, instilled in to the afflicted eye conjunctival sac with insulin to prevent further formation of new blood vessels, and shrink the existing pathologically formed blood vessels and reduce the edema in wet AMD. This method incorporates putting the patients on low fat diet, aerobic exercise, ketamine-a NMDA blocker, reducing the blood cholesterol using adjuvant therapeutic agents selected from Statins, that are inhibitors of 3-hydroxy-3-methylglutaryl coenzyme A, (i.e. HMG-Co A) reductase which in turn reduce drusen formation that leads to AMD, combined with insulin ophthalmic drops.

### CROSS REFERENCE TO RELATED APPLICATIONS

This is a continuation in part of U.S. patent application Ser. No. 12/940,247, filed Nov. 5, 2010 the complete disclosure is hereby incorporated by reference.

### FIELD OF THE INVENTION

This invention relates to the treatment of age related macular degenerative (AMD) diseases of the retina affecting the vision in humans or the animals.

### BACKGROUND OF THE INVENTION

Age related macular degeneration (AMD) is a retinal eye disease that affects the macula lutea with fovea centralis involved in central vision. This is the most common cause of blindness. The fovea centralis of the macula is a small spot in the central area of the retina located at the back of the eye. The macula is responsible for sight in the centre of the field of vision.

Structure of the Fovea: To understand the AMD, it is important know the histological structure of the Fovea. The center of the fovea is known as the foveal pit (Polyak S L. The retina. Chicago: University of Chicago Press; 1941) and is a highly specialized region of the retina different from adjacent central and peripheral retina. It is the Radial small circular region of retina measuring less than a quarter of a millimeter (200 microns) across. The foveal pit is an area where cone photoreceptors are concentrated at maximum density with exclusion of the rods, and arranged at their most efficient packing density, which is in a hexagonal mosaic. Below this central 200 micron diameter central foveal pit, the other layers of the retina are displaced concentrically leaving only the thinnest sheet of retina consisting of the cone cells, RPE, Bruch's membrane and choroid. Radially distorted but complete layering of the retina then appears gradually along the foveal slope until the rim of the fovea, which is made up of the displaced second- and third-order neurons related to the central cones. Here the ganglion cells are piled into six layers, making this area, called the foveal rim or parafovea (Polyak SL.IBID), the thickest portion of the entire retina.

The complete foveal area including foveal pit, foveal slope, parafovea, and perifovea considered the macula of the human eye (FIGS. 4.5.6). This area is familiar to ophthalmologists is a yellow pigmentation to the macular area known as the macula lutea. This pigmentation is the reflection from yellow screening pigments, the xanthophyll carotenoids zeaxanthin, and lutei (Balashov N A, Bernstein P S. Purification and identification of the components of the human macular carotenoid metabolism pathways. Invest Ophthal V is Sci. 1998; 39:s38.), present in the cone axons of the Henle fiber layer. The macula lutea is thought to act as a short wavelength filter, additional to that provided by the lens (Rodieck R W. The vertebrate retina: principles of structure and function. San Francisco: W.H. Freeman and Company; 1973.). The flourescein angiography of this area show a ring of blood vessels in the macular area around a blood vessel- and capillary-free zone 450-600 um in diameter, denoting the fovea.

The macular blood vessels arise from branches of the superior temporal and inferotemporal arteries. At the border of the avascular zone, the capillaries become two layered and finally join as a single layered ring. The collecting venules are more deep (posterior) to the arterioles and drain blood flow back into the main veins (Zhang H R. Scanning electron-microscopic study of corrosion casts on retinal and choroidal angioarchitecture in man and animals. Prog Ret Eye Res. 1994; 13:243-270). In the rhesus monkeys, this perimacular ring and blood vessel free fovea is clearly seen in the picturesque striking drawings made by Max Snodderly's and his partners (Snodderly D M, Weinhaus R S, Choi J C. Neural-vascular relationships in central retina of Macaque monkeys (Macaca fascicularis). J. Neurosci. 1992; 12:1169-1193). As the fovea is the most essential part of the retina for human vision, protective mechanisms for avoiding bright light and especially ultraviolet irradiation damage are essential. For, if the delicate cones of fovea are destroyed, blindness ensures.

The above described foveal cone photoreceptors are affected in AMD. Symptoms of AMD depend upon the stage of the AMD. The most common symptom comprises straight lines in the field of vision appears wavy. The type in books, magazines, and newspapers appears blurry. The dark or empty spaces block the centre of vision. Troubles reading street signs, doing things at home or work because the lights seem dimmer, Trouble recognizing the faces of friends and family, trouble with close work such as reading, sewing or picking out matching clothes, Diminished color intensity, Difficulty adapting to low light, especially for sensitive vision tasks like reading.

The AMD can be “Nonexudative” “dry” macular degeneration and “Exudative” “Wet” macular degeneration. These two kinds may have one or more of the following abnormal findings such as “Geographic atrophy”, “Retinal Pigment Abnormalities”, “Detachment of the RPE”, “Choroidal Neovascularization (CNV, SRNVM)” and “Loss of Vision” as being symptomatic of macular degeneration cured or curtailed or prevented from progressing.

AMD is associated with: Drusen: Pigmentary alterations, Exudative changes: hemorrhages in the eye, hard exudates, subretinal/sub-RPE/intraretinal fluid (FIG. 7); Atrophy: incipient and geographic in which the Visual acuity drastically decreased (Example: 20/20 to 20/80 vision or worst); Blurred vision: Those with nonexudative macular degeneration (dry type) may be asymptomatic or notice a gradual loss of central vision. Whereas those with exudative macular degeneration (wet type) often notice a rapid onset of vision loss and central scotomas (shadows or missing areas of vision), Distorted vision i.e., metamorphopsia)—a grid of straight lines appears wavy and parts of the grid may appear blank. Patients often first notice this when looking at mini-blinds in their home and trouble discerning colors; specifically dark ones from dark ones and light ones from light ones. Slow recovery of visual function after exposure to bright light. A loss in contrast sensitivity and Preferential hyperacuity perimetry changes are seen in wet AMD.

People with age related macular degeneration might find difficulty in doing simple everyday activities requiring sharp vision. In the United States, macular degeneration affects over 13 million people. AMD is the leading cause of visual impairment for persons age 75 and older (30% affected). Above the age of 65, individuals lose at least 10% of their central vision resulting in the visual impairment related to the development of macular degeneration. Macular degeneration affects 1 in 10 people over the age of 65, as the average age of the U.S. population continues to increase so does the number of people suffering from AMD. More than 200,000 new cases develop annually. AMD is more common in non-Hispanic whites than in blacks or Mexican-Americans. According to the forecast, Age-Related Macular Degeneration cases will increase from 13 million in 2010 to 17.8 million by 2050. In non-vitamin-receiving individuals, cases of choroidal neovascularization (CNV) with geographic atrophy increased from 1.7 million in 2010 to 3.8 million by 2050. In us, it is estimated that the cases of visual impairment and blindness will increase from 620,000 in 2010 to 1.6 million in 2050 when given no treatment (David B. Rein, et al; for the Vision Health Cost-Effectiveness Study Group The Potential Impact of New Treatments Arch Ophthalmol. 2009; 127(4):533-540).

AMD affect the macula lutea that comprises only about 2.1% of the retina, and the remaining 97.9% (the peripheral field) remains unaffected by the disease. Interestingly, even though the macula provides such a small fraction of the visual field, almost half of the visual cortex is devoted to processing macular information. The loss of central vision profoundly affects visual functioning. It is not possible, for example, to read without central vision. Pictures that attempt to depict the central visual loss of macular degeneration with a black spot do not really do justice to the devastating nature of the visual loss.

What causes AMD is unknown. There are factors which can increase the risk of developing AMD such as: genetics—a family history of macular degeneration, being, female, possess a light skin tone, widespread exposure to UV light, high blood pressure, Aging—an estimated 10% of AMD are under the age of 50, Diabetes, elevated total serum cholesterol, higher body mass index (BMI), and Smoking. The smoking has consistently been associated with higher AMD risk compared to other risk factors.

Wanda Hamilton, the Executive Director of AMD Alliance International, spell out that smoking and genetics play the greatest roles in determining if you may be at risk of developing AMD. “If you have a particular gene make-up and you smoke, you could be up to 144 times more likely to get AMD. If you have other genes and you smoke, you could be up to seven times more likely than non-smokers to get the disease.” The reason being, cataract removal creates a higher risk for AMD with the removal of the lens allows previously filtered light to pass unobstructed to the retina. At times Transition lenses, also, called photochromic lenses prescribed for AMD for this reason. These lenses change from nearly clear indoors to darker outdoors. This type of lens cuts the glare and provides clarity of vision and comfort for someone with macular degeneration. Ophthalmologists perform dilated eye exams, ophthalmoscopic exam, fluorescein angiograms, and use Amsler grids as well as other tests to diagnose AMD.

There are measures that one can take to reduce the risk of AMD. The following health measures may prevent, delay, or curtail the onset and the effects of AMD. They are as follows: Do not smoke, Always wear sunglasses (use both blue and UV light blocking glasses) even on cloudy days and in the winter, wear hats and decrease your exposure to the Sun. The individual needs to keep the blood pressure and cholesterol at the proper level, to keep weight at a healthy level by Exercise for 30 minutes at least four times weekly to help maintain ideal body weight and optimal blood pressure and aerobic initiated circulation to tissues. The reduction dietary fat to 20-25% of total dietary calories, decrease red meats, whole milk, cheese, and butter while increasing consumption of omega-3 fatty acids (e.g., cold-water fish, canola oil, etc.) reduce the incidence or delay the development of AMD, The individual needs to consume abundance of fruits and vegetables, especially green, leafy ones such as Kale, spinach. Reduction consuming of junk food (processed foods) and eat two or more servings of fish which are high in omega 3 every week like salmon and mackerel is in order. Living a healthy lifestyle and lifelong UV protection are essential to reducing ones risk of developing AMD.

Simple natural dietary habits reduce the risk of developing AMD. Lutein, Vitamins A, C, and E all offer benefits for overall eye health. Take vitamin C (500 mg), vitamin E (400 IU), beta-carotene (15 mg) or vitamin A, and zinc (80 mg as zinc oxide), daily. Vitamin A can help to reduce the risks of cataracts and night blindness. The deficiency of Vitamin A implicated in blindness and corneal ulcers. Vitamin C reduces pressure in glaucoma, slows age-macular related degeneration (AMD), and prevents cataracts. Vitamin C is a powerful antioxidant that is highly concentrated in the lens of the eye. Vitamin E helps to reduce the risk of macular degeneration and cataracts. These supplements have not been shown to prevent AMD; however, these supplements slow the progression of the established disease. Two important antioxidants for eye health that must be in the diet are lutein and zeaxanthin. They are found in leafy, green vegetables such as spinach, kale and fresh parsley, yellow fruits and vegetables. Minerals needed to help the body metabolize vitamins, balance nutrition, and hormones. Critical minerals for photoreceptors health include zinc and selenium.

Other important supplements for eye health are lutein, bioflavonoids, and carotenoid. Natural supplements for eye health should include bilberry and blueberry, which contains antioxidant compounds that help maintain the strength and the structure of eye capillaries and retina. The grape seed extract is a natural powerful antioxidant. Proanthocyanidins recommended for their powerful vascular strengthening abilities and antioxidant activity. Blood sugar kept normal. The patient should avoid MSG, hydrogenated oils, artificial food flavoring, and coloring agents. Smokers should avoid taking beta-carotene (Age Related Eye Disease Study Research Group. A randomized, placebo-controlled, clinical trial of high-dose supplementation with vitamins C and E, beta carotene and zinc for age related macular degeneration and vision loss Arch Ophthalmol 2001; 119:1417-36). The patient needs to eat more green leafy vegetables and supplement with use of lutein-zeaxanthin supplements. These pigments help to reduce the effects of blue light as it penetrates the macula and RPE.

AMD affects the macula lutea (FIGS. 4, 5, 6). The area of the macula comprises only about 2.1% of the retina, and the remaining 97.9% (the peripheral field) remains unaffected by the disease. The center of the macula called the fovea centralis, the area of location for the cones photoreceptors. There are no rods located in the fovea centralis. The fovea is the place of sharpest and most sensitive visual acuity. Macula is a highly specialized retina located at the back of the eye directly facing the center of the cornea and lens. It is responsible for sight in the centre of the field of vision. Macula is approximately an eighth of an inch in diameter. The macula has densely packed photoreceptors cone photoreceptors that collect light which are responsible for central vision. The peripheral retina is composed mainly rods, which are the light-sensitive cells responsible for side and night vision. The macula is one hundred times more sensitive to detail than the peripheral retina. The human macula has 7 million special cones in each eye and a dense concentration of ganglion cells. They permit high resolution of visual acuity compared to 110-120 million rods involved in the peripheral and dark vision, in the rest of the retina in each eye.

In a healthy macula, the clear layer of the retina on the inside of the eye nourished and maintained by the retinal pigment epithelium (RPE). Behind the pigment epithelium is the non-cellular Bruch's membranous layer and vascular choroid, which contains the rich net work of blood vessels and choroidal lamellar cells (between the choirdal BV and Sclera). These are the extension of the pia-arachnoid membrane of the optic nerve. These cells layers have spaces in between to transport tissue fluid and nourishment to, and carry out metabolic waste away from the retina (FIGS. 4-7) (Shantha T R and Bourne G H: Histological and Histochemical studies of the choroid of the eye and its relations to the pia-arachnoid mater of the central nervous system and Perineural epithelium of the peripheral nervous system. Acta Anat 61:379-398 (1965). Shantha T. R. and Bourne G H. Arachnoid villi in the optic nerve of man and monkey. Expt Eye Res 3:31-35 (1964)).

Three forms of macular degeneration identified, and they are: 1. atrophic, non-exudative-dry form occurs in 85 to 90% of patients with macular degeneration. 2. Exudative commonly known as wet form occurs in 10% of patients usually treated with laser surgery; and 3. Pigment epithelial detachment associated (PED) AMD occurs in less than 5% of the patients resulting in retinal detachment. In the dry form, there is a breakdown or thinning of the retinal pigment epithelial cells (RPE) in the macula, hence the term “atrophy”. These RPE cells are important for the proper functioning of the retina. They metabolically support the overlying photoreceptor. In the wet form of macular-degeneration, abnormal blood vessels grow uncontrolled called subretinal neo-vascularization (SRNV) under the retina. They lift the retina up with loss of ability to see (FIG. 7).

In the normal choroid, the large blood vessels (BV) have intact thick vessel walls. The choriocapillaries coming out of the main choroidal BV have fenestrations or openings in their walls allowing easily the contents of the circulating blood to leak out to the extracellular Bruch's membranous space on the surface of RPE in turn supplies nutrient to the underlying retinal photoreceptors cells (FIGS. 3,4,5,7). In patients with AMD, new blood vessels proliferate from these choriocapillaries through Bruch's membrane adjacent to the retinal pigment epithelium (RPE), and form a mass of vascular plexus (FIG. 6). The resulting choroidal neovascularizations (new vessels in the choroid) occur with around 10% of the patients with AMD. Such choroidal neovascularizations go with other oculopathies such as diabetic retinopathy, pathologic myopia, ocular histoplasmosis syndrome, and other idiopathic conditions. The fluid from these BV (blood, cellular elements, electrolytes, plasma fluid, drugs in plasma if the person on medications orally or as ophthalmic drops) leaks to the surrounding tissue. This fluid can increase, build up pressure, and press on the RPE and retina, resulting in their detachment leading to defective vision and blindness (FIG. 7).

Ultimately, the fluid may be absorbed and drying which leads to scarring. In the dry type of AMD, the RPE cells die resulting atrophic AMD. As AMD advances, the person loses the sharp, central vision needed to see straight ahead and to engage in such activities as reading, needlework and driving. With no appropriate treatment, many of them become legally blind in both types of AMD. This condition is the leading cause of loss vision in US above the age sixty years or older.

In “dry” macular degeneration, there is a slow breakdown of photoreceptors cone reducing central vision. About 90 percent of people with macular degeneration have this dry form. Treatment with additional supplemental vitamins and minerals may slow the progress of the disease. As “dry” macular degeneration worsens, new, fragile blood vessels (BV) grow beneath the macula from the choroid above the pigment layer. The dead photoreceptors neurons allow the BV to grow (angiogenic). The cones may be anti angiogenic and their destruction results in continued unabated angiogenesis leading to the pathology. These new blood vessels often leak blood and fluid, which causes further damage to the macula, leads to loss of central vision-what is known as “wet” macular degeneration (wet AMD—FIG. 7).

Wet AMD treatment consists of laser surgery or Photodynamic therapy to destroy new blood vessels. Only about 15 percent of patients with the “wet” form of macular degeneration are suitable for laser surgery because the new blood vessels grow too close to the macula where the visual image focused. Laser treatment only applied after sight-threatening changes have occurred. In spite of laser treatment, the disease and loss of vision may progress unabated. The loss of vision is permanent and can't be restored. No medical treatment is currently available that can be both prophylactic and prevent for macular degeneration hence we bring this new method of treatment. We call the AMD “The diabetes of the eye”

Retinal pigment epithelial cells (RPE) are virtually black due to melanin pigment, which is similar to hair pigment. They form a layer that recharges the photoreceptor cells of the eye after they are exposed to light. The photoreceptors contain molecules called photopigments in their outer segments in close proximity to the photoreceptors. When light (photons) strikes these molecules, they absorb the light and change shape (uncoiling), sending a signal to the brain indicating they have “seen” light. Once a photopigment molecule absorbs light, it needs to be recharged. The photopigment molecule is shuttled out of the photoreceptor and down to the RPE cells. The RPE cells recharge the photopigment molecules and send them back to the photoreceptors outer segments to start the process again. This process takes 20 minutes. In addition, the RPE layer keeps the photoreceptors healthy by collecting, storing, and disposing toxic waste products produced during the process of regenerating the photopigment during light perception. In macular degeneration for reasons that are not yet completely obvious, the RPE cells are unable to provide this support for the photoreceptors and both of these cells eventually die. Microscopic studies of the atrophic cells in senile macular degeneration patients (post mortem) show retinal pigment epithelium cellular elements, destroyed with the pigment clumped and adhered to the undersurface of the Bruch's membrane. These studies suggest an inflammatory process induced by a degradation product or irritant in the area of the destroyed retinal cells. That is why the Macular degeneration of the retina is a progressive degeneration of the pigmented cells and subsequent destruction of the cone photoreceptors of the retina of unknown etiology.

Interestingly, the retina has a similar topographical layer arrangement of cytoarchitecture to the brain; it is an extension of the brain and winnow to the brain. The six layers of the retina carry the function of transmitting light stimuli into the brain through the optic nerve. Then through the brainstem structure of the lateral geniculate, the optic radiates to the occipital lobe sensory neurons. The layers of the retina consists of a neuro-ectodermal layer of rods and cones, an intermediate layer of bipolar cells, horizontal cells and Muller's cells, and the inner layers containing ganglion cells, glia, nerve fibers, and internal limiting membrane separated from the choroid by retinal pigment epithelium (RPE, FIGS. 8,9).

The rods and cones are the photoreceptors of the retina. They consist of photoreceptive pigment and inner segments with dense packing of mitochondria like folded sheet. Besides retina, the pigmented cells occur in the red nucleus, substantia nigra, and locus coeruleus in the brain. These pigmented cells of the retina are hexagonal cells lying just externally to the rods and cones layer of the retina. These cells provide insulation of melanin pigment, nutrition and provide the Vitamin A substrate for the photosensitive pigments in the rod and cone cells.

Patients with an early stage of AMD are diagnosed by the occurrence of anomalous clumps of irregular pigments in the eye examination namely Drusen (FIG. 7). The first visible defect in AMD is buildup of drusen, a lipoproteinaceous deposit between RPE and Bruch's membrane, the extra cellular matrix between the RPE and the underlying choroid. Drusen are a significant risk factor for the progression to choroidal neovascularization (CNV), the most important cause of vision loss in AMD (FIG. 7). The presence of large, soft drusen in the eye indicates a pre-stage of exudative AMD, and places patients at higher-than-average risk for developing neovascularizations (FIG. 7).

As noted, the loss of central vision in macular degeneration is due to the atrophy of the retinal pigment epithelium (RPE) associated with loss of cone retinal photoreceptors. There have been reports of histiocytes and giant cells in the areas of breaks in Bruch's membrane (which acts as outer blood retinal barrier) and subretinal neovascular membranes. The RPE transports metabolic waste from the photoreceptors across Bruch's membrane to the choroid. Bruch's membrane gets thicker (up to 3 times the normal) with advancing age. This impedes the transportation of waste material that can cause a buildup of deposits and can also contribute to AMD patho-physiology. The development of drusen may be the result of this clogging of the transport system of the BV at the periphery of the macula lutea. The lipoprotein—cholesterol fat—cellular derbies and calcium deposits continue to accumulate with formation of drusen similar to athermanous patch in the BV.

These built up deposits formed on and in Bruch's Membrane are called: 1. Basal Linear Deposits (BLinD) and 2. Basal Lamellar Deposits (BLAMD). The deposits cause breakdown of this membrane and allows the choroid vessels to burst through and to expand into the membrane and RPE where it is beyond the retina itself. In choroidal neovascularization (CNV), capillaries coming from the choroid must cross Bruch's membrane to reach the subretinal pigment epithelial space. Studies show that the “Human Bruch's membrane ages like arterial intima and basement membrane” and the plasma lipoproteins are the known source of extracellular cholesterol. Hence the “Age-related maculopathy and atherosclerotic cardiovascular disease (ASVD) may share joint pathogenic mechanisms”

How AMD interrelated to systemic ASVD further supported by the study of what people eat fatty diet, obese and who develops AMD, night blindness and heart disease. The following studies do support the food we eat and development of AMD with ASVD. Besides lutein and vitamin A, supplements to treat night blindness, how the inflammation and cholesterol plays a role in development of AMD that can lead to night blindness described herein. The discovery of macular degeneration gene (CPH gene variant is involved in regulating the inflammatory pathways) lends support to this hypothesis. Recent research provides additional support. High blood levels of two biomarkers of inflammation—C-reactive protein (CRP) and interleukin 6 (IL-6)—are associated with a twofold increase in the risk of progression of macular degeneration that is associated with night blindness so also the risk of ASVD. More than 1 serving/week of beef, pork, or lamb as a main dish is associated with a 35% increased risk of macular degeneration compared with less than 3 servings/month. A high intake of margarine is also significantly related to an increased risk of AMD. 1 serving per day of high-fat dairy food (whole milk, ice cream, hard cheese, or butter) increases risk of macular degeneration progression by 1.91 times. 1 serving per day of meat food (hamburger, hot dogs, processed meat, bacon, beef as a sandwich, or beef as a main dish) increases risk of macular degeneration progression by 2.09 times. 1 serving per day of processed baked goods (commercial pie, cake, cookies, and potato chips) increases risk of macular degeneration progression by 2.42 times. People who eat fish more than 4 times/week have a lower risk of macular degeneration than those who consume it less than 3 times/month. This is especially true for Tuna fish. People who eat canned tuna more than once per week are 40% less likely to develop macular degeneration as compared with those who consumed it less than once per month. Fish is a major source of DHA (an omega-3 fatty acid). Recently it has been reported that there is a potential beneficial effect of eating any type of nuts on risk of progression of macular degeneration. Eating 1 serving per day of any type of nut reduces the risk of progression of macular degeneration by 40%. This beneficial effect complements other literature reporting a protective role for nuts and cardiovascular disease and type 2 diabetes mellitus. One of the bioactive compounds in nuts, resveratrol, has antioxidant, antithrombotic, and anti-inflammatory properties. We advised all our patients' vegetable diet with fish and less red meat and dairy products. As prophylactic method, all our AMD and aged patients with ASVD risks are changed into a regimen of fish, vegetable, nuts with least red meat, and minimal dairy products diet.

The retinal layers supplied by two vascular systems. Retinal vessels from the central artery of the retina (a branch of the ophthalmic artery) supply the inner two-thirds. The outer retina is completely avascular which receives oxygen and nutrients from the choroidal BV. To enhance transport of oxygen and nutrients and to remove the metabolites from the photoreceptors, there is a major pool of fenestrated choroidal capillaries beneath the retina. This pool referred to as the choriocapillaris.

Plasma and other constituents leak out of the choriocapillaris to pools beneath the retinal-pigmented epithelium (RPE), which has tight junctions with several transport systems. This constitutes the outer blood-retinal barrier through the Bruch's membrane. Inner Retinal vascular endothelial cells have tight junctions, which creates the inner blood-retinal barrier. The inner limiting membrane (ILM) lines the inner surface of the retina and the peripheral borders of the vitreous, which is also avascular. The inner retina is a vascularized tissue sandwiched between two avascular tissues, which the outer retina is an avascular tissue pack in between two vascularized tissues.

The unique architecture of the retina makes the possibility to identify two types of neovascularization: First, retinal neovascularization, which sprouts from retinal vessels, penetrates the Inner Liming Membrane (ILM) and grows into the vitreous (although, under some circumstances, the vessels grow the other way through the avascular outer retina to the subretinal space). Second, Choroidal Neochoriocapillares (CNV), which sprouts from choroidal vessels, penetrates Bruch's membrane and grows in the sub RPE and subretinal spaces (FIG. 7) (Campochiaro P. A., Retinal and Choroidal Neovascularization, Journal of cellular Physiology 184:301-310, 2000).

Blood vessels develop by vasculogenesis, angiogenesis, or intussusception. During vasculogenesis, the endothelial cells of the BV differentiate from precursor cells and the angioblasts are already present throughout the tissue, where there is linkage in concert to form vessels. During angiogenesis, BV germinates from preexisting BV and invades into surrounding tissue that we see in AMD (FIGS. 7-9). Most organs are vascularized by vasculogenesis, except, the brain and parts of the kidney. Retinal vascular development occurs by a combination of vasculogenesis (new BV) and angiogenesis from existing BV (McLeod D S, Lutty G A, Wajer S D, Flower R W. 1987. Visualization of a developing vasculature. Microvasc Res 33:257-269. McLeod D S, Crone S N, Lutty G A. 1996. Vasoproliferation in the neonatal dog model of oxygen-induced retinopathy. Invest Ophthalmol V is Sci 37:1322-1333.). Superficial retinal vessels formed by vasculogenesis.

Angiogenesis plays an important role in pathogenesis of wet AMD, diabetic retinopathy and many eye diseases as well as other systemic diseases including cancers. Hence, it is important to understand the pathophysiology of this process, to understand the effect of various pharmacological and therapeutic anti angiogenesis agents for the treatment of AMD. U.S. Pat. No. 6,525,019 B2 discloses melanin based therapeutic agents for inhibition of angiogenesis of AMD. There are many specific antiangiogenesis monoclonal antibodies developed to block the abnormal genesis of BV, which we use with insulin as part of our invention.

Abnormal angiogenesis is the most common cause of blindness and is involved in approximately twenty eye diseases. Such angiogenic damage is associated with diabetic retinopathy, retinopathy of prematurity, corneal graft rejection, neovascular glaucoma, and retrolental fibroplasias, AMD etc. The only known angiogenesis inhibitors which specifically inhibit endothelial cell proliferation are angiostatin protein and Endostatin™ protein (O'Reilly M. S., Holmgren L., Shing Y., Chen C., Rosenthal R. A., Cao Y., Moses M., Lane W. S., Sage E. H., Folkman J. Angiostatin: a circulating endothelial cell inhibitor that suppresses angiogenesis and tumor growth. Cold Spring Harbor Symp. Quant. Biol., 59: 471-482, 1994. O'Reilly M. S., Boehm T., Shing Y., Fukai N., Vasios G., Lane W. S., Flynn E., Birkhead J. R., Olsen B. R., Folkman J. Endostatin: an endogenous inhibitor of angiogenesis and tumor growth. Cell, 88: 277-285, 1997. Yoon S. S., Eto H., Lin C. M., Nakamura H., Pawlik T. M., Song S. U., Tanabe K. K. Mouse endostatin inhibits the formation of lung and liver metastases. Cancer Res., 59: 6251-6256, 1999. Dhanabal M., Ramchandran R., Waterman M. J., Lu H., Knebelmann B., Segal M., Sukhatme V. P. Endostatin induces endothelial cell apoptosis. J. Biol. Chem., 274: 11721-11726, 1999.). Thus, the new methods and ophthalmic drops compositions are needed that are capable of inhibiting angiogenesis and treating angiogenesis-dependent diseases like wet AMD and the other angiogenesis related diseases of the eye and other parts of the body. Such antiangiogenesis effects augmented—amplified by the use of our invention in conjunction.

Individuals with lighter iris color develop higher incidence of age related macular degeneration (AMD) than those with darker iris color. (Frank R N, Puklin J E, Stock C, Canter L A (2000). “Race, iris color, and age related macular degeneration”. Trans Am Ophthalmol Soc 98: 109-15; discussion 115-7). Evidence indicates that individuals with increased iris pigmentation have a decreased risk of developing AMD. The increased levels of eumelanin appear to be more protective than pheomelanin and the light-absorbing characteristics of melanin are thought to be responsible for this protective effect (Hammond B R, Jr, Fuld K, Snodderly D M. Iris color, and macular pigment optical density. Exp Eye Res. 1996; 62:293-297).

An alternative hypothesis is that increased levels of melanin may protect against age related increases in lipofuscin (implicated in photo-oxidative mechanisms). However, these prior studies do not teach, discuss, or suggest the antiangiogenic ability of melanin to inhibit blood vessel growth and macular degeneration, as disclosed in the invention U.S. Pat. No. 6,525,019 B2. According to the present invention, melanin, and melanin-promoting compound, applied in combination with other compositions and procedures for the treatment of AMD. The melanin, or melanin-promoting compound, formulations includes those suitable for oral, ophthalmic (including intravitreal or intracorneal or conjunctival sac), nasal, topical (including buccal and sublingual), and other parenteral routes. Our invention of using insulin promotes melnogenesis in the RPE, hence prevent or curtail angiogenesis in AMD.

U.S. Pat. No. 6,936,043 B2, and U.S. Pat. No. 6,942,655 B2 disclose using PDT to treat AMD and may need many treatments, which can further damage the retina. PDT prevents or alters the function of the neovascular tissue by using low energy light to generate reactive species within the vessels, or within and around the vessels, thereby damage these vessels and prevent further growth.

U.S. Patent Application Pub. No.: 2003/0065020 A I, discloses a method of treating or preventing macular AMD by administering an HMG-CoA reductase inhibitor. It is based on the finding that men and women who use statins are associated with an 11-fold reduction in risk of macular degeneration. Statins are inhibitors of 3-hydroxy-3-methylglutaryl coenzyme A, i.e. HMG-CoA reductase inhibitors. Accordingly, we provide that age related macular degeneration (AMD) is effectively treated by administration of HMG-CoA reductase inhibitors like statins comprising: fluvastatin (Lescol), cerivastatin (Baycol), atorvastatin (Lipitor), imvastatin (Zocor), pravastatin (Pravachol), lovastatin (Mevacor) and rosuvastatin (ZD 4522). They provide a method of treating AMD by: (a) lowering the level of LDL cholesterol in the patient; (b) increasing the level of HDL cholesterol in the patient; and (c) lowering the level of triglycerides in the patient's blood.

Other HMG-CoA reductase inhibitors are disclosed in U.S. Pat. No. 6,218,403, U.S. Pat. No. RE 36,481 and U.S. Pat. No. RE 36,520 U.S. Pat. Nos. 5,877,208, 5,792,461 and 5,763,414 disclose the use of naringin and naringenin, citrus peel extract and hesperidin and hesperetin respectively as HMG-CoA reductase inhibitors. These incorporated with our invention of insulin to treat AMD.

U.S. Pat. No. 6,218,403, U.S. Pat. No. RE 36,481 and U.S. Pat. No. RE 36,520 U.S. Pat. Nos. 5,877,208, 5,792,461 and 5,763,414 discloses a method of treating age related macular degeneration with a therapeutic amount of a prostaglandin F₂ₐ from derivative like latanoprost. This method is based on the property of prostaglandin F₂ₐ derivatives cause the iris and other tissues to darken when applied topically to the eye. This may increase the melanin and reduce the AMD when used in conjunction with our invention topically.

A novel process for making latanoprost taught in U.S. Pat. No. 5,466,833 and the use of latanoprost in treating glaucoma are disclosed in U.S. Pat. No. 5,510,383. It is known that prostaglandin F derivatives have the ability to stimulate melanogenesis in tissues, which they are applied as described in U.S. Pat. No. 5,905,091. The application of latanoprost to the eye during the treatment of glaucoma results in increased pigmentation of the eye when light-colored eyes with blue irises can change to brown irises. This effect of prostaglandin F₂ₐ derivatives is discussed in the drug insert for the latanoprost ophthalmic solution from Pharmacia & Upjohn. This melanogenistic Property has been seen as a negative side effect of the use of prostaglandin F₂ₐ derivatives. It is suggested treatment be discontinued if increased pigmentation ensues during treatment. Solutions to overcome this problem disclosed in U.S. Pat. No. 5,886,035. In AMD, the melanogenesis factor is taken as positive to restore the function of the RPE and treat AMD.

U.S. Pat. No. 6,525,019 B2 discloses the therapeutic agent melanin for inhibition of angiogenesis of AMD. Melanin located within specific cells called melanocytes. Melanin present in the skin, hairs, and eyes where they impart the color and play a role in light absorption that acts as free-radical scavenger (antioxidant).

U.S. Pat. No. 2,145,869 by Dr. Donato Perez Garcia disclose a method for the treatment of syphilis in general and neurosyphilis in particular using subcutaneous insulin injections followed by intravenous infusion of arsenic, mercury, and bismuth, therapeutic agents with glucose and calcium chloride.

U.S. Pat. No. 4,196,196 discloses a composition of insulin, glucose and magnesium dipotassium ethylene diamine tetra acetic acid (EDTA) to enhance tissue perfusion and to facilitate a divalent/monovalent cation gradient uptake in and out of the cells. Insulin in the intravenous infusion with glucose enhances the uptake and activity of potassium and magnesium at the extra and intra cellular level that is well established.

I have used this method for decades in many surgical and post surgical patients that have other diseases to alter the potassium level in the extracellular fluid (blood) and intracellular levels of the cells, whenever, there was low or high levels of potassium in the serum.

U.S. Pat. No. 4,971,951 and U.S. Pat. No. 5,155,096 discloses Insulin Potentiation Therapy (IPT) for the treatment of virally related diseases such as hepatitis and AIDS, Gonorrhea, duodenal ulcer, gall stones, epilepsy, schizophrenia, asthma, arthritis, osteomyelitis, cancers, and many other disease conditions using insulin. These inventions do not describes the use of insulin locally to treat age related macular degeneration or any other retinal diseases or other local disease condition of the other organs as described in this invention.

None of these inventors and patents discloses or describes the local (topical) or regional tissue or organ specific use of insulin in dry AMD, and insulin with monoclonal antibodies in wet AMD in a restricted area of the tissue or organ to treat the disease states described herein. Regrettably, now, there is no effective way to treat dry or wet form of age related macular degeneration. Unfortunately, no dry AMD treatment breakthrough achieved yet. We believe that our invention will be a breakthrough to cure or curtail dry AMD. The insulin and monoclonal antibodies will maintain the integrity of RPE and photoreceptors, prevent further loss, and induce mitosis in the remaining healthy RPE cells. The inventive method described herein is simple and noninvasive procedure without any adverse effects.

### SUMMARY OF THE INVENTION

A method for treating age related macular degeneration (AMD) using an insulin preparation applied topically to the conjunctival sac of the affected eye. Another aspect of this invention is using antiangiogenic adjuvant therapeutic agents such as bevacizumab, ranibizumab, pegaptanib, etanercept, instilled in to the afflicted eye conjunctival sac with insulin to prevent further formation of new blood vessels, and shrink the existing pathologically formed blood vessels and reduce the edema in wet AMD. This method incorporates putting the patients on low fat diet, aerobic exercise, ketamine-a NMDA blocker, reducing the blood cholesterol using adjuvant therapeutic agents selected from Statins, that are inhibitors of 3-hydroxy-3-methylglutaryl coenzyme A, (i.e. HMG-Co A) reductase which in turn reduce drusen formation that leads to AMD, combined with insulin ophthalmic drops.

The present invention describes the AMD, development, types, signs, symptoms, pathophysiology and treatments available and new modalities of treatment described in this invention.

One aspect of the present invention is a method for treating the age related macular degeneration in humans or animals by administering insulin to the afflicted eye.

Another aspect of the present invention is a method for treating the AMD in humans or animals by administering to the afflicted eye by insulin combined with various known adjuvant therapeutic agents, as well as other nurticeuticals, pharmaceuticals, biochemical, and biological agents or compounds.

The present invention furthermore uses this method as a prophylactic on patients where the patients are predisposed to develop Age related macular degeneration (AMD) with hypercholesterimia treated with statins.

The present invention additionally relates to treatment of other oculopathies associated with or contributing to age related macular degeneration.

The present invention uses insulin to stimulate the retinal pigment epithelium to maintain proper functioning of the RPE, and Bruch's membrane, and photoreceptors in dry AMD.

The present invention uses insulin in its various forms to induce mitogenesis of stem cells in the RPE-retinal complex, or the embryonic stem cells introduced intra-vitreal and maintain the health of the retina.

The present invention uses insulin to stimulate the Bruch's membrane to function properly, and to maintain its' integrity, which prevents the growth of choroidal capillary into the RPE, and to act as effective choroid retina barrier.

The present invention uses insulin to augment and amplify the effects other adjuvant therapeutic agents many times, so that small dose of the toxic or expensive therapeutic agents needed to treat AMD.

The present invention uses tetracycline and its derivatives, rifamycin and its derivatives, macrolides, and metronidazole, with insulin prevents the formation and the destruction of formed capillaries.

The present invention discloses a method and apparatus for effectively administering a natural enzyme lipase (lipoprotein lipase) into the posterior sclera in close proximity to the macula that will dissolve lipid deposits in the body of the membrane and assist in their removal through the choroidal circulation, along with insulin to enhance health of the RPE, Retina, and choroid BV.

The present invention uses medication comprising lutein and zeaxanthin, antioxidants or a mixture thereof that are tailored to an individual by providing an effective amount of a carotenoid and/or vitamin C, vitamin E; beta carotene, zinc and/or a mixture to said subject, with insulin to enhance health of the RPE, photoreceptors and choroidal capillaries.

The present invention is used to treat all forms of wet age related macular degeneration by administering topiramate with a pharmaceutically effective dosage to suppress degeneration or induce growth of new optic nerve fibers over a sustained period along with insulin to enhance health of the RPE, Retina and choroidal capillaries.

The present invention is for use with all forms of wet, age related macular degeneration by the administration of a topical application of non-steroidal anti-inflammatory agents (NSAID) along with insulin to enhance health of the RPE, Retina, and choroidal capillaries and prevent angiogenesis.

The present invention is for use with all forms of age related macular degeneration by administration of Triamcinolone acetonide, prednisone; para, beta or dexamethasone, and related corticosteroids with insulin.

The present invention of is for use with all forms of wet age related macular degeneration by administration of topical application of carbonic anhydrase inhibitors to the eye such as dorzolamide, acetazolamide, methazolamide and other compounds along with insulin to enhance health of the RPE, Retina and choroidal capillaries and reduce the chances of edema in wet AMD.

The present invention is for use with all forms of age related macular degeneration by administration of a topical application of with a adjuvant therapeutic amount of a prostaglandin F₂ₐ, derivative such as latanoprost along with insulin to enhance health of the RPE, Retina and choroidal capillaries by increasing the melanin content which is antiangiogenic.

Another aspect according to the present invention, a method of using a pharmaceutically acceptable carrier insulin for an HMG-CoA reductase inhibitor for the treatment or prevention of macular degeneration and to prevent Drusen formation.

Preferably, the HMG-CoA reductase inhibitor comprises a statin selected from the group consisting of: fluvastatin (Lescol™), cerivastatin (Baycol™), atorvastatin (Lipitor™), simvastatin (Zocor™), pravastatin (Pravachol™), lovastatin (Mevacor™) and rosuvastatin (ZD 4522) administered in combination with insulin ophthalmic drops to enhance their uptake in the ocular vascular tissue. The combination of ophthalmic insulin drops with HMG-CoA reductase inhibitor act by (a) lowering the level of LDL cholesterol; (b) increasing the level of HDL cholesterol; and (c) lowering the level of triglycerides in the patient resulting in the reduction or further formation of Drusen in the macula of the eye.

Intent of the present invention is, a method of using pharmaceutically acceptable carrier insulin for an HMG-CoA reductase inhibitor for the treatment or prevention of macular degeneration; to prevent formation and progression of Drusen formation. Drusen cause loss or decrease of visual acuity, deformation of vision, loss of central vision, choroidal neovascularisation (CNV) to develop, progression from dry to wet form, geographic atrophy, RPE degeneration and detachment; sub retinal or sub-RPE hemorrhage and sub-RPE fibrous tissue formation (FIG. 7). The present invention prevents Drusen formation resulting in prevent and progression to above pathology.

Intent of this invention is to prevent macular degeneration in a second healthy eye from developing or progressing in a patient having a established macular degeneration in one eye.

Another object of this invention directed to a method to prevent, alleviate, or delay the onset of AMD and to reduce further loss of vision in a patient having AMD.

Another object of this invention directed to a method to prevent, alleviate, or delay the onset of AMD and to reduce further loss of vision in a patient having AMD by blocking the excitotoxic effect of glutamate on photoreceptors by using ketamine as NMDA blocker.

The invention directed to a method to reduce the recurrence of new vessels by administering monoclonal antibodies with insulin in an eye of a patient having undergone laser coagulation therapy for AMD by further treating the patient with PDT concomitantly with laser coagulation therapy.

The present invention provides methods and compositions for treating diseases and processes mediated by undesired and uncontrolled angiogenesis by administering to a human or animal with a composition with insulin comprising melanin, melanin-promoting compound, and Bevacizumab, Ranibizumab, Pegaptanib monoclonal antibodies and protein complexes.

It is intent of this invention to provide insulin ophthalmic drops to enhance the health and multiplication of stem cells injected intravitreal, extracted from the human embryo to treat dry AMD. Insulin is a trophic factor needed for multiplication and various biological activities of the stem cells so as to seed the RPE stem cell and promote their take at RPE.

Another broad object of this invention to apply insulin ophthalmic drops along with the following therapies published experimentally in multiple patents to treat AMD for curing or curtailing AMD. The following are some of the experimental therapies published, where insulin can be incorporated in addition to their therapeutic agent's inventions.
a) U.S. Pat. No. 5,948,801 discloses the use of Brinzolamide as eye drops.
b) U.S. Pat. No. 6,716,835 B1 discloses a method of retarding degeneration of retinal photoreceptors in patient afflicted with age-related macular degeneration using calcium channel blocker compounds and/or cyclic GMP-dependent channels, namely diltiazem, for treating retinal pathologies, and more particularly retinal diseases caused by degeneration of visual receptors.
c) U.S. Patent Application Publication Number: 2001/0049369 AI demonstrates that brimonidine tartrate, a potent alpha-2 adrenergic receptor agonist, applied topically to the eyes can prevent photoreceptor cell degeneration. The Muller cell associated with degenerative signs in an in vitro model of retinal degeneration and retinal detachment. Brimonidine allowed for the formation of highly structured photoreceptor outer segments, prevented the expression of stress markers in Muller cells, and preserved the expression patterns of Muller cell markers of proper cell-to-cell contact and differentiation. Using this adjuvant therapeutic agents with insulin descried in our invention will enhance its therapeutic effects and prevent the angiogenesis.
d) Mitoxantrone (Novantrone) is a chemotherapeutic drug that the drug works by suppressing the immune system. This can inhibit the vascular growth in wet AMD when used as ophthalmic drops.
e) Omega 3 fatty acids include Alpha-linolenic acid (ALA), Eicosapentaenoic acid (EPA), and Docosahexaenoic acid (DHA). The Omega 6 fatty acids include Linoleic acid (LA), Gamma linolenic acid (GLA), Dihomo-gamma-linolenic acid (DGLA), and Arachidonic acid (AA). Gamma-linolenic acid (GLA) is an omega-6 fatty acid found mostly in plant-based oils. GLA is considered an essential fatty acids and antioxidants essential for macular health.
f) Follow the instruction as described in the above EXAMPLE 1. A method of topically instilling insulin drops to a person or animals' conjunctival sac to treat age related macular degeneration with administration of insulin. The insulin enhances their uptake. The insulin has therapeutic activity by entering into afflicted structures in the eye. This can be combined with uptake facilitators such electroporation, iontophoresis, sonophoresis, vibroacoustic, vibration, and other physical (heat, magnetic force, radio frequency, microwave, laser lights etc.) methods with other appropriate adjuvant therapeutic, biological, pharmacological anti-glaucoma, and retinal protectors. These agents combined with insulin therapy as described. These methods can be used as prophylaxis, to diagnose, prevent and to treat the above conditions.
g) U.S. Pat. No. 6,525,019 B2 discloses the therapeutic agent melanin for inhibition of angiogenesis of AMD. Melanin located within specific cells called melanocytes. Melanin can be enhanced by insulin ophthalmic drops which can prevent the development of angiogenesis. Individuals with lighter iris color have been found to have a higher incidence of age-related macular degeneration (AMD) than those with darker iris color. (Frank R N, Puklin J E, Stock C, Canter L A (2000). “Race, iris color, and age-related macular degeneration”. Trans Am Ophthalmol Soc 98: 109-15; discussion 115-7).
h) U.S. Patent Application Pub. No: 2005/0239757 A1 disclose methods for treating AMD and other degenerative ocular condition using progesterone which can be used also with insulin ophthalmic drops.
i) U.S. Pat. No. 4,656,188 discloses the angiotensin converting enzyme inhibitors (ACE inhibitors) are useful in the treatment of senile macular degeneration. Their discovery based that the senile macular degeneration is a poorly characterized disease state of the elderly, which appears to result from a poor blood supply to the macular region of the eye. ACE inhibitors dilate the retinal BV, and their effect is augmented by addition of ophthalmic drops in addition.
j) U.S. PATENT APPLICATION PUB. NO.: 200710037782 A1 disclose the therapeutic agent for aging macular degeneration comprises a progesterone derivative with special formulation.
k) Other drugs, like sunitinib (Sutent®) and sorafenib (Nexavar®), are small molecules that attach to the VEGF receptor. This keeps it from being turned on and making new blood vessels. Some drugs already used to treat cancer have been found to inhibit the blood vessel growth. They can be effective in wet AMD with insulin.
l) U.S. Patent Application Pub. No.: 200910155381 A1 determine the susceptibility to AMD, then use medication comprising lutein (wherein the carotenoid is lutein and/or zeaxanthin) and/or zeaxanthin and/or certain antioxidants (or a mixture thereof)
m) U.S. Pat. No. 5,314,909 discloses the topical application of non-steroidal anti inflammatory agents (NSAID) to treat AMD. There is a well documented effect of Indomethacin in the treatment of cystoid macular edema. Senile macular degeneration has an increased permeability of the retinal capillaries and some destruction of retinal pigment epithelium. They disclose the use of indomethacin, diclofenac, ketorolac, flurbiprofen, and the like to treat this condition. Combining with insulin can enhance their effect. We used Cox-2 inhibitors in all our cancer patients to prevent the angiogenesis and metastasis.
n) U.S. Pat. No. 6,046,223 discloses a method for treating and/or preventing macular edema and age related macular degeneration which comprises topical administration of carbonic anhydrase inhibitors to the eye such as Dorzolamide, acetazolamide, methazolamide, and other compounds which are described in U.S. Pat. Nos. 5,153,192; 5,300,499; 4,797,413; 4,386,098; 4,416,890 and 4,426,388.
o) Dawson et al. describe that the Pigment epithelium derived factor is potent (PEDF) inhibitor of angiogenesis (Dawson D. W., Volpert O. V., Gillis P., Crawford S. E., Xu H., Benedict W., Bouck N. P. Pigment epithelium-derived factor: a potent inhibitor of angiogenesis. Science (Washington D.C.), 285: 245-248, 1999). Volpret et al. describe the anti angiogenic effect of Interleukin-4 (Volpert O. V., Fong T., Koch A. E., Peterson J. D., Waltenbaugh C., Tepper R. I., Bouck N. P. Inhibition of angiogenesis by interleukin 4. J. Exp. Med., 188:1039-1046, 1998.). Thus the PEGF and interleukin-4 can be used in AMD with our invention to prevent, curtail, or cure the condition.
p) Deferoxamine is a chelating agent used to remove excess iron from the body. Iron removed which the reduction reduces the damage done to various organs and tissues, like the liver, CNS, and retina. The damage that we saw in the retina can be due to excessive iron from the choroid and retinal blood vessels leaking excessive iron reacting with ROS, where the excess damages the sensitive photoreceptors. Deferoxamine ophthalmic drops with insulin can remove excess iron at macula lutea, reduce ROS damage, and prevent angiogenesis and wet AMD.

### BRIEF DESCRIPTION OF THE DRAWINGS

FIG. 1 is a schematic representation of the longitudinal section of the eye 100 showing conjunctival sac 202 where the ophthalmic preparation of this invention, the insulin, monoclonal antibodies drops are instilled into the conjunctival sac.

FIG. 2 is a schematic representation of the longitudinal section of the eye 200 showing the structures involved in the production and the drainage of aqueous humor which and the structures that collect, and transport the therapeutic agents including insulin, used in the treatment of AMD of this invention.

FIG. 3 is a schematic representation of the anterior part of the eye 300 presenting the rich vascular plexus that are responsible for transporting the insulin and other adjuvant therapeutic agents to the macula and the rest of the retinal photoreceptors.

FIG. 4 is a schematic diagram of the sagittal section of the eye 400 and the location of the macula lutea.

FIG. 5 is a schematic diagram of the longitudinal section of the eye 500 and the location of the macula lutea.

FIG. 6 is a diagrammatic presentation showing the rich vascular plexus of the uveal system.

FIG. 7 is a schematic view of the longitudinal section of the part of the eye and the location of wet AMD.

FIG. 8 is a schematic representation showing the histology of the retina in relation to the blood supply and to delineate how AMD develops and therapeutic agents of this invention reach the site of pathology.

FIG. 9 is a diagrammatic presentation showing the histology of the external layers of retina including photoreceptors.

FIG. 10 is a diagrammatic presentation showing the route of drainage of the lacrimal fluid and therapeutic agents and how to prevent nasal mucosal uptake.

### DETAILED DESCRIPTION OF THE INVENTION

Terms used: As used in this document, the terms “macular degeneration”, “age-related macular degeneration”, and “age-related maculopathy”, as well as the abbreviations “AMD”, “ARMD”, “ARM” are synonymous and used interchanging. The ophthalmic drops or preparations used to treat age related macular degeneration should be stable, dissolved, or solubilized which the preparation is safe and effective with ophthalmological standards in place, Preferably in the aqueous composition without the particulate, crystalline, or droplet form in the composition. The term ‘stable’, means physical, rather than chemical stability with no crystallization and/or precipitation in the compositions, when the preparation is stored at a refrigerated or room temperature. The preparation encounters lacrimal secretions when the preparation applied to the conjunctival sac and the cornea, and should not react with it. The phrase “ophthalmological acceptable” refers to those therapeutic, pharmaceutical, biochemical and biological agents or compounds, materials, compositions, and/or dosage forms suitable for use in a mammalian eye without undue toxicity, irritation, allergic response, or other problem or complication, commensurate with a reasonable benefit/risk ratio. The expression ‘safe and effective’ means a concentration and composition that the concentration and composition is sufficient to treat without serious local or systemic side effects. Our invention fulfills all these parameters used with ophthalmic drops to treat AMD. The term “ocupopathies” means all diseases affecting the eyelids, eyeball with retina, optic nerve, choroid, eyeball as whole, and their function. “Therapeutic agents” means the various known therapeutic agents, as well as other pharmaceutical, biochemical, nurticeuticals, and biological agents or compounds which are effective in the treatment of AMD. “Augmentation and amplification” effects mean the enhancement of uptake and amplification of therapeutic effect once inside the afflicted cells. “Adjuvant” means in addition to primary agent of our invention, it names the other already known therapeutic agents to have curative or curtailing effect on the ARD. The term “drop” “drops” means the therapeutic agents delivered or instilled to conjunctional sac by a dropper or plastic squeeze bottle drop by drop. The ophthalmic drops or preparations used to treat AMD should be stable, dissolved, or solubilized which the preparation is safe and effective with ophthalmological standards in place. The terms “instilled” and “applied” used interchanging.

The terms “treat,” “treating” and “treatment” “cure” “Curtail” used herein, and unless otherwise specified, which reduces, retards, slows the progression and the severity of the disease using the invention and therapeutic agents described herein.

In the following detailed description of the invention, reference made to the drawings in which reference numerals refer to like elements, and intended to show by way of illustration specific embodiments in which the invention may be able to treat AMD. It is understood that other embodiments may be utilized and that structural changes may be made without departing from the scope and spirit of the invention.

The following diagrams describes the structure of the eye, and explains the route of movement, transportation, and diffusion of insulin, monoclonal antibodies, and other adjuvant therapeutic agents instilled in the conjunctival sac topically for the treatment of AMD.

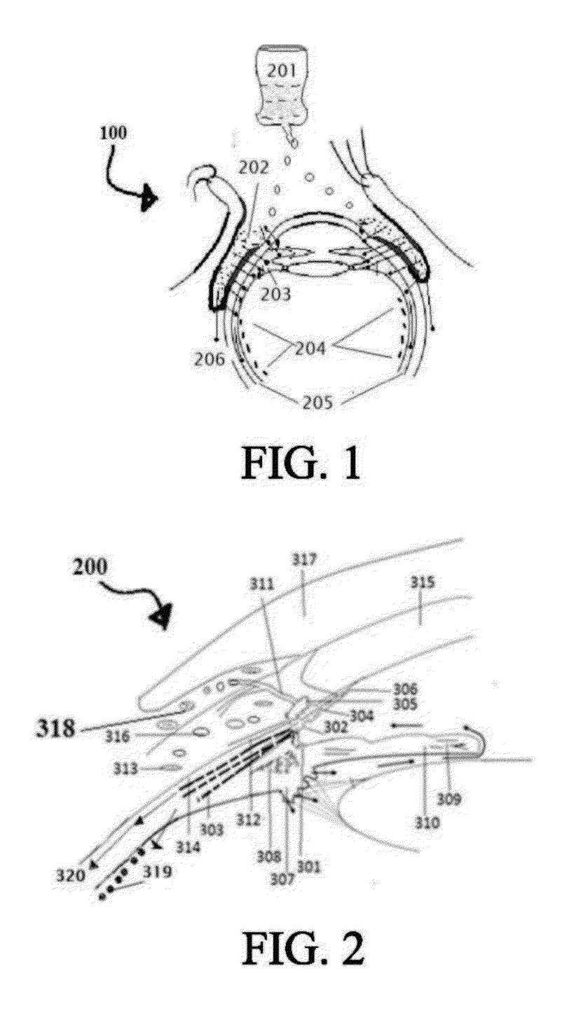

FIG. 1 is a schematic representation of the longitudinal section of the eye 100 showing conjunctival sac 202 where the ophthalmic preparation of this invention, the insulin, monoclonal antibodies drops are instilled into the conjunctival sac. The therapeutic agents introduced through a dropper 201 and their passage to iridocorneal angle, anterior and posterior chambers, iris, ciliary body, and processes 203, choroid, and the anterior segment of the retina 204 that contains photoreceptors rods and cones (macula lutea) that the photoreceptors affected by the AMD (drumstick markers). Note that the ophthalmic insulin, Monoclonal antibodies eye drops, and other adjuvant therapeutic agents pass on to the choroid 205 adjacent to the retinal pigment epithelium and retinal outer segment of the photoreceptors, delivers the therapeutic agents to the afflicted rods and cones. The therapeutic agents passes through the episcleral plexus of veins to the periphery of the sclera 206, from where the therapeutic agents can be reabsorbed and circulate back into the choroid and retinal blood vessels (BV).

FIG. 2 is a schematic representation of the longitudinal section of the eye 200 showing the structures involved in the production and the drainage of aqueous humor which and the structures that collect, and transport the therapeutic agents including insulin, used in the treatment of AMD of this invention. The insulin circulates through various sites of action where the therapeutic agents reach their ultimate site of action with ease to the retinal cones in the macula lutea and rods (arrows). The therapeutic agents entering the anterior chamber aqueous humor transported through the episcleral arteriovenous plexus 313,316, 318. Then pass through the uveoscleral meshwork 301, corneoscleral meshwork 302, Juxtacanalicular or cribriform trabecular meshwork 304, Schlemm's canal 305, Corneal endothelium joining the trabecular meshwork 306, Longitudinal 303, and circular fibers of the ciliary muscles 308; muscle fibers of the iris 309, 310, Scleral sinus vein 311, Scleral Spur 312, Scleral Veins 313,316, Suprachoroidal space between choroid and sclera 314. The cornea 315 and sclera 316 participate the least in therapeutic agent's circulation or transport except at the cornea-scleral junction. The conjunctival sac 317 (fornix) where the insulin, and other therapeutic agents or compounds are deposited to be transported (arrows) to the retina through the ciliary body 307, trabecular mesh work, choroid, and irido-scleral angle 301, choroid plexus projecting from the ciliary body 307. The choroid plays an important role in transporting the insulin, and other adjuvant therapeutic agents (arrows) to the AMD afflicted cones and retinan 319 (From Shantha T R and Bourne G H. Some observations on the corneal endothelium. Acta Ophthalmologica 41: 683-688: 1963).

This diagram illustrates the ease with which the insulin, monoclonal antibodies, and other selected therapeutic agents of our invention reach the afflicted photoreceptors 319 site from the conjunctival sac (arrows) of this invention. From the conjunctival sac 317, the therapeutic agents enter into the anterior chamber, corneal endothelium 306, 304, trabecular meshwork 301, 302, and ciliary body 308, passing through the sub and inter conjunctival blood vessel plexus of the eye 313, 316, 318. Then the therapeutic agents transported to the choroid 320, suprachoroidal space 314 where they reach their destination 319 to have therapeutic effect on the macula lutea and retina involved in AMD. This diagram also shows how simple, physically and physiologically uncomplicated it is for the therapeutic agents of this invention to reach the choriocapillaries, pigment epithelium, Bruch's membrane, and macula lutea, which are the site of major pathology in dry and wet AMD. The arrows markers indicate the site of entry and the circulation of the insulin, monoclonal antibodies, and adjuvant therapeutic agents from the conjunctival sac where they exert their effect in the treatment of AMD.

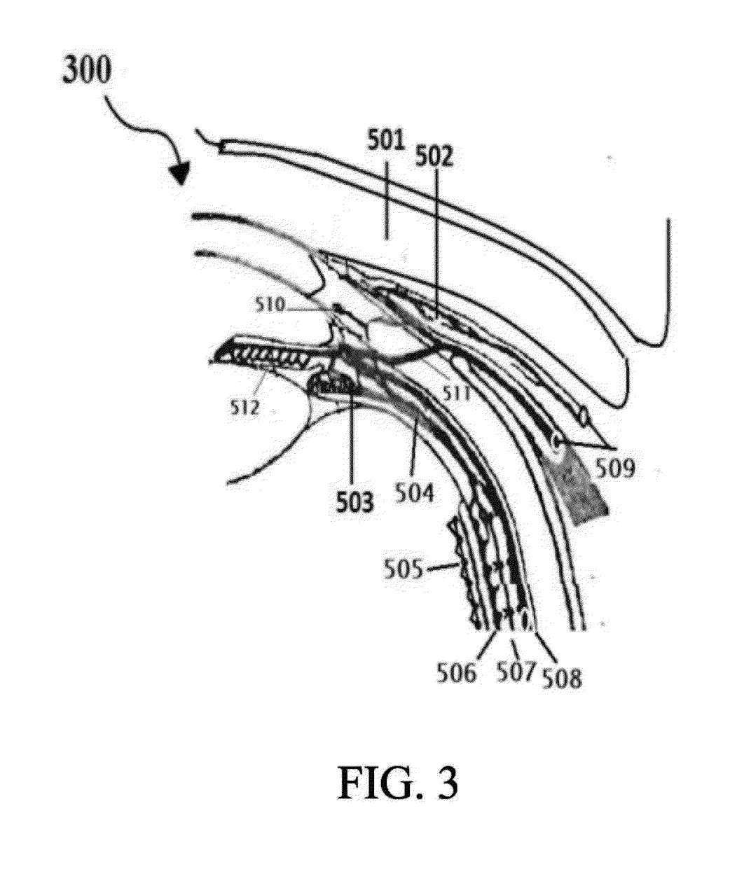

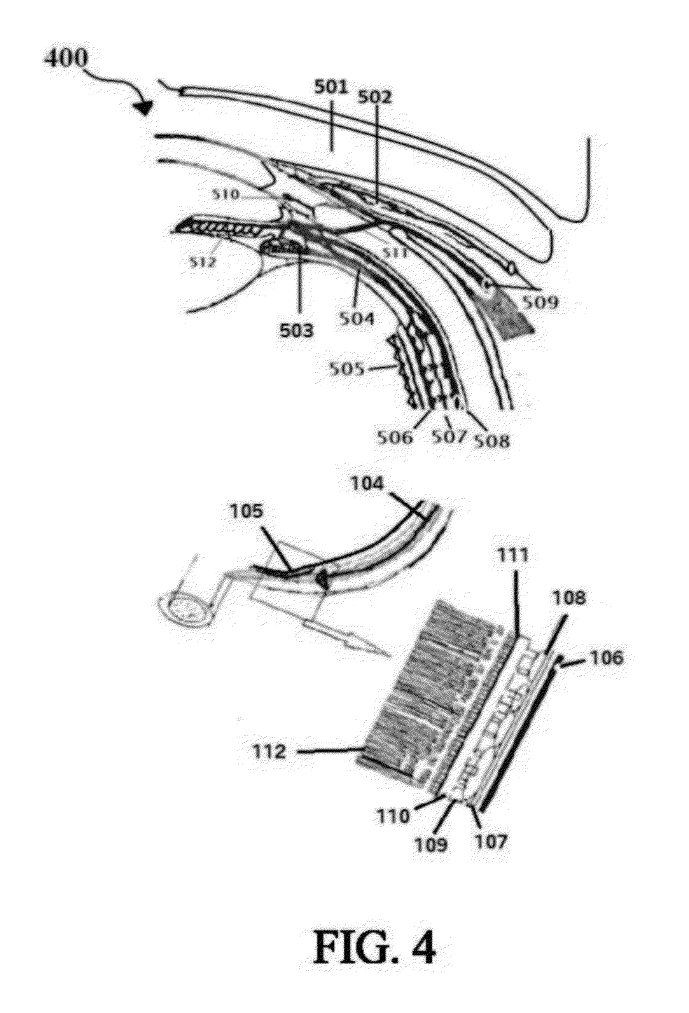

FIG. 3 is a schematic representation of the anterior part of the eye 300 presenting the rich vascular plexus that are responsible for transporting the insulin, Monoclonal antibodies and other therapeutic agents of this invention from the conjunctival sac 501 to the rods 505 and macula lutea posteriorly (see FIG. 4,5). Note the rich vascular plexus 502 under the conjunctiva of the eye that transport the therapeutic agents from the conjunctival sac 501. The therapeutic agents from these sites pass through the intrascleral 511 veins and canal of Schlemm 510. They are connected with the other BV and various vascular structures of iris 512, iridocorneal angle, ciliary body with the ciliary processes 503 where there are rich BV, and finally passes to the choroid vascular plexus 504, 507, Bruch's membrane, retinal pigment epithelium 506, supra and inter choroidal space 508. From here, the therapeutic agents reach the base of the rods 505 and macula lutea of the retina, the site of the AMD. Note the rich vascular plexus of the iris 512, choroid, ciliary body 503. These BV communicates with the subconjunctival BV 502, suprachoroidal space 508, and choroidal vascular net work 504,507. The choroidal vascular network delivers insulin, Monoclonal antibodies and anti AMD therapeutic agents to various structures between the ciliary body and the iridoslceral angle and scleral-corneal space, and supra scleral network of vascular plexus 509 finally reaching the RPE and retina.

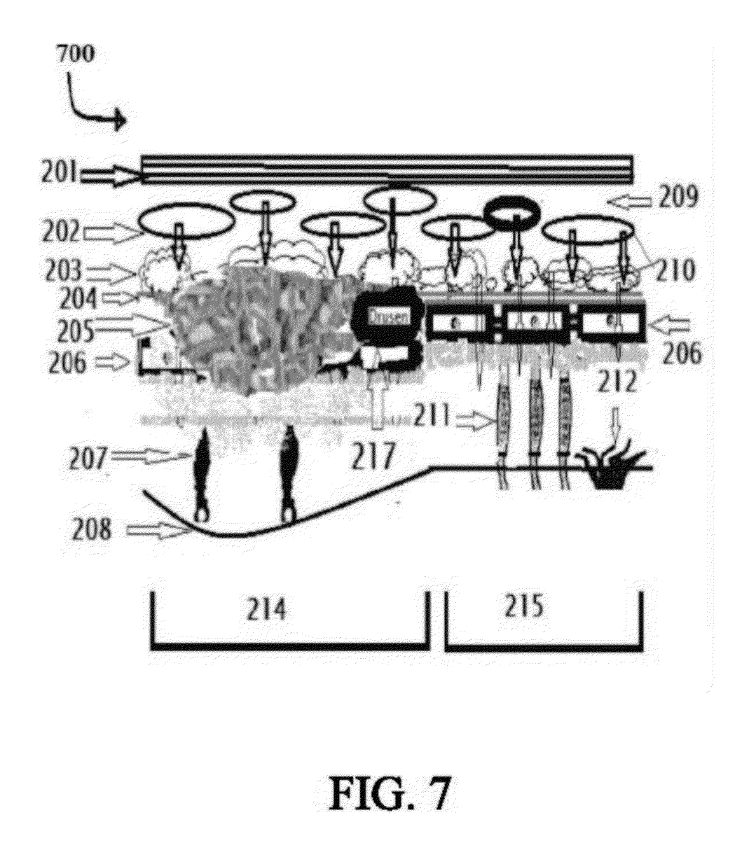

FIG. 4, is a schematic diagram of the sagittal section of the eye 400 and the location of the macula lutea 105 (boxed in) and its histological structures 106-112 affected by the AMD. The rest of the explanations are same as in FIG. 3. A diagram is showing the route of delivery of Insulin and other adjuvant therapeutic agents to the macula, the site of AMD from the conjunctival sac. From the conjunctival sac 102 the therapeutic agents are absorbed by choroidal vascular system 104 through the subconjunctival BV, intrascleral blood vessels and transported to the choirdal BV 104 and suprachoroidal space 107. They reach the macula lutea 105 and fovea centralis (boxed space). The insulin and other therapeutic agents including monoclonal antibodies from the conjunctival sac reaches the choroidal BV 108 below the suprachoroidal space 107 and sclera 106. From these large BV of the choroid 108, the insulin and other therapeutic agents enter the fenestrated choriocapillaries 109 (See FIG. 7). The insulin leaks through the choriocapillaries 109 to Bruch's membrane 110 and transported to pigment epithelium 111, which may be the primary site of pathology, then to the photoreceptors 112 of the fovea centralis and the structures surrounding the fovea and macula lutea.

The therapeutic agents deposited in the conjunctival sac 501, enters the anterior chamber aqueous humor through the episcleral arteriovenous plexus. Then pass through the uveoscleral meshwork, Corneoscleral meshwork, Juxtacanalicular or cribriform trabecular meshwork, Schlemm's canal, Corneal endothelium, joining the trabecular meshwork, Longitudinal and circular fibers of the ciliary muscles; muscle fibers of the iris, Scleral sinus vein, Scleral Veins, Suprachoroidal space 107, spaces between choroidal lamellae and sclera 107. The conjunctival sac 502 (fornix), where the therapeutic, pharmaceutical, biochemical and biological agents or compounds are deposited to be transported to the Macula Lutea 105 (boxed in) and its histological contents (arrow) 106-112 of the retina. The therapeutic agents pass through the anterior chamber, irido-scleral angle, ciliary body, choroid plexus projecting from the ciliary body, choroid 104, play an important role in transporting the insulin, monoclonal antibodies and other therapeutic agents to the Macula, the site of AMD. This diagram illustrates how easy it is for the insulin and other selected therapeutic agents to reach the afflicted AMD site 105 from the conjunctival sac 502. This method therapeutic agent's delivery prevents the therapeutic agents circulating all over the body through the systemic circulation to reach the site of AMD with their associated adverse effects if taken orally or parentarily.

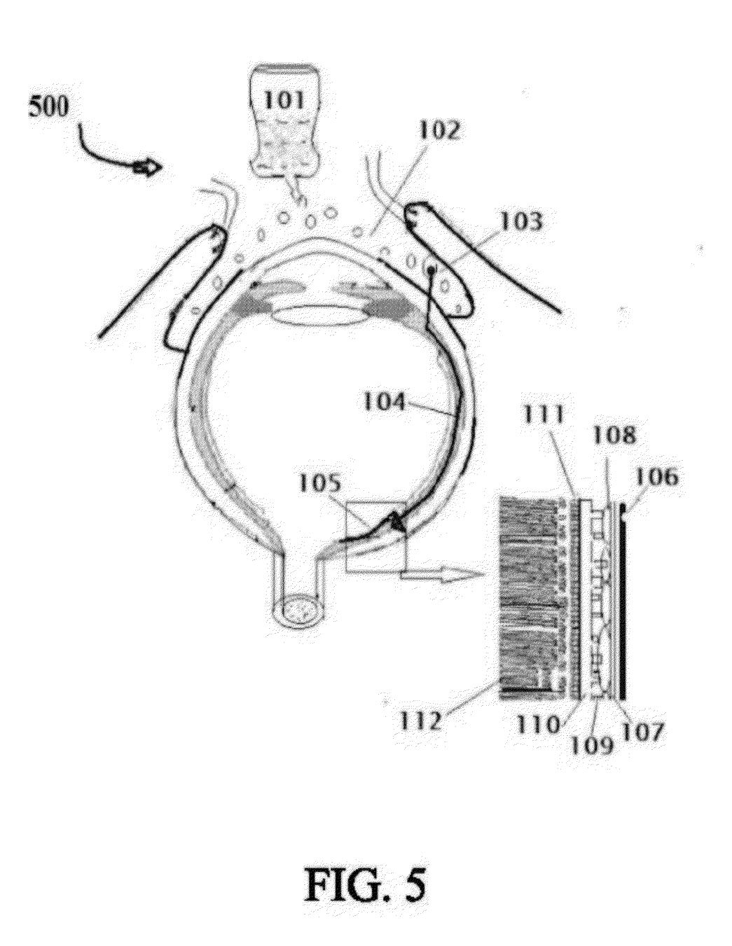

FIG. 5 is a schematic diagram of the longitudinal section of the eye 500 and the location of the macula lutea 105 (boxed in) and its histological structures 106-112 affected by the AMD. A diagram is showing the route of delivery of Insulin and other therapeutic agents to the macula, the site of AMD from the conjunctival sac. It shows the eyedropper 101 applying the therapeutic agents to the conjunctival sac 102. From the conjunctival sac 102, the therapeutic agents 103 are absorbed by choroidal vascular system 104, through the subconjunctival BV, intrascleral blood vessels. From there, the therapeutic agents are transported to the choirdal BV 104 and suprachoroidal space 107. They reach the macula lutea 105 and fovea centralis (boxed space) passing through the Bruch's membrane and RPE. The insulin from the conjunctival sac reaches the choroidal BV 108 below the suprachoroidal space 107, between the layers of choroidal lamellae, and sclera 106. From these large BV of the choroid 108, the insulin and other therapeutic agents enter the fenestrated choriocapillaries 109. The insulin and monoclonal antibodies also permeate through the choriocapillaries 109 to Bruch's membrane 110 and transported to pigment epithelium 111 to the photoreceptors 112 of the fovea centralis and the structures surrounding the fovea and macula lutea and to the rest of the retinal photoreceptors.

The therapeutic agents 103 deposited in the conjunctival sac enter the anterior chamber aqueous humor through the episcleral arteriovenous plexus. Then pass through the uveoscleral meshwork, Corneoscleral meshwork, Juxtacanalicular or cribriform trabecular meshwork, Schlemm's canal, Corneal endothelium joining the trabecular meshwork, Longitudinal and circular fibers of the ciliary muscles; muscle fibers of the iris, Scleral sinus vein, Scleral Veins, Suprachoroidal space between choroid and sclera 107 (FIGS. 3 and 4). The therapeutic, pharmaceutical, biochemical and biological agents or compounds described in this invention are deposited the conjunctival sac 102 (fornix). From this location, they are transported to the Macula Lutea 105 (boxed in) and its histological contents (arrow) 106-112 of the retina. To reach this site of action, the therapeutic agents are passing through the anterior chamber, irido-scleral angle, ciliary body, choroid plexus projecting from the ciliary body, and choroid 104. They all play an important role in transporting the insulin, monoclonal antibodies, and other therapeutic agents to the Macula 105, the site of AMD pathology. This diagram illustrates how easy it is for the insulin, monoclonal antibodies and other selected therapeutic agents to reach the afflicted AMD site 105 from the conjunctival sac 102. The arrow marker 103 indicate the site of entry of therapeutic agents through various above described structures of the anterior segment of the eye to be effective in the treatment of AMD acting to prevent, further progression, and curing the AMD. This method therapeutic agent's delivery prevents the therapeutic agents circulating all over the body through the systemic circulation to reach the site of AMD'S with their associated adverse systemic effects if taken orally or parentarily.

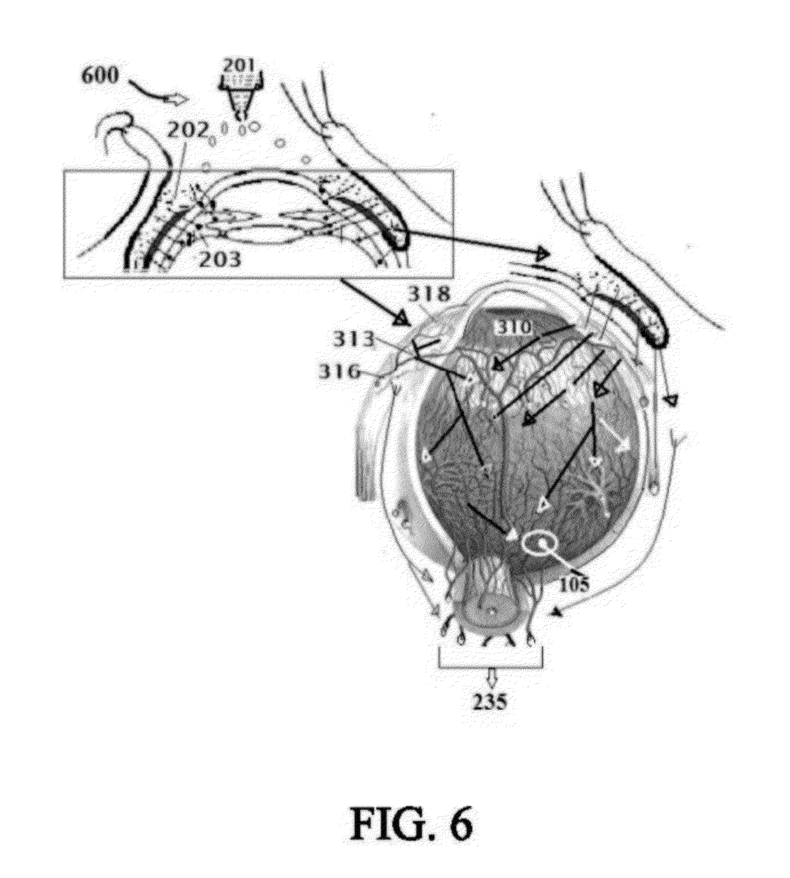

FIG. 6 is a diagrammatic presentation 600 showing the vascular plexus of the uveal system. The uveail system and its rich BV plays an important role in the transport of insulin, monoclonal antibodies and therapeutic agents delivered to conjunctional sac 202. The uveal system or track is the middle layer of the eye, divided from front to back into, the iris 310, ciliary body 203, and the choroid (arrows) covering the entire retina which are involved in the transport of insulin, Monoclonal antibodies and other therapeutic agents of this invention to the retina, and the sites of the AMD. These three structures of the uveal system are vascular and they communicate with the subconjunctival 318 and scleral vessels 313,316, 318. The entire uvea is drenched with aqueous humor, which permeates between the choroidal lamellae and suprachoroidal space (Shantha T R and Bourne G H: Histological and histochemical studies of the choroid of the eye and its relations to the pia-arachnoid mater of the central nervous system and Perineural epithelium of the peripheral nervous system. Acta Anat 61:379-398 (1965). Shantha T. R. Shantha, and Bourne G H: Arachnoid villi in the optic nerve of man and monkey. Expt Eye Res 3:31-35 (1964)). Based on the Shantha studies (IBID), there is constant to and fro of flow of fluid from the anterior chamber of the eye and subarachnoid space (SAS) CSF through the lamina cribrosa into the choroid. The insulin, Monoclonal antibodies and the adjuvant therapeutic agents 201 from the conjunctival sac 202 are transported to the sub conjunctival venous plexus 318 inter and epi scleral veins 313,316, 318, then these therapeutic agents are transported to the uveal vascular plexus (multiple drumstick and plain arrows). Through this rich vascular plexus, the therapeutic agents reach the outer segment of photoreceptors of the retina and macula lutea 105, that are located immediately adjacent to the choroid situated on the retinal pigment epithelium.

The blood vessels of the uveal system are involved in the health of the retina by transporting and by providing proper nurticeuticals; oxygen, at the same time, the products of metabolites removed from these photoreceptors. In the same fashion, they carry insulin and monoclonal antibodies, and the adjuvant therapeutic agents, and deliver to the retinal cones, and RPE, the site of AMD 105. This diagram shows, how efficiently the insulin, Monoclonal antibodies and the other therapeutic agents from the conjunctival sac 202 are absorbed and transported to the subconjunctival, scleral vascular plexus 318, 313,316; then delivered to the uveal system (arrows) including iris 310, ciliary body and then to the retina, the site of AMD pathology. Arrows points to the spread of therapeutic agents from the conjunctival sac to the rich choroidal vascular network. There is no other organ in the body that is surrounded by such a complex rich vascular network. Long curved arrows shows that some of the therapeutic agents are transported to the supra scleral space where the agents may be transported back through the penetrating arterio-venous net work on the optic nerve (arrows) and posterior surface of the sclera (Based on Grays Anatomy diagram 7.255 on the histology of the eye).

FIG. 7 is a schematic view of the longitudinal section of the part of the eye 700 and the location of the macula lutea 214 and its histological structures in wet AMD compared to healthy retina 215. This diagram shows the location and pathology of the wet AMD in the retina, pigment epithelium, and choroidal blood vessels (BV). The diagram shows the pathology of the AMD of the fovea centralis 214 compared to the rest of the healthy retina 215. The diagram shows the sclera 201, large BV of the choroid 202 and the choriocapillaries 203 and 210. Note the invasion of the choroidal neochoriocapillares 205 (CNV)

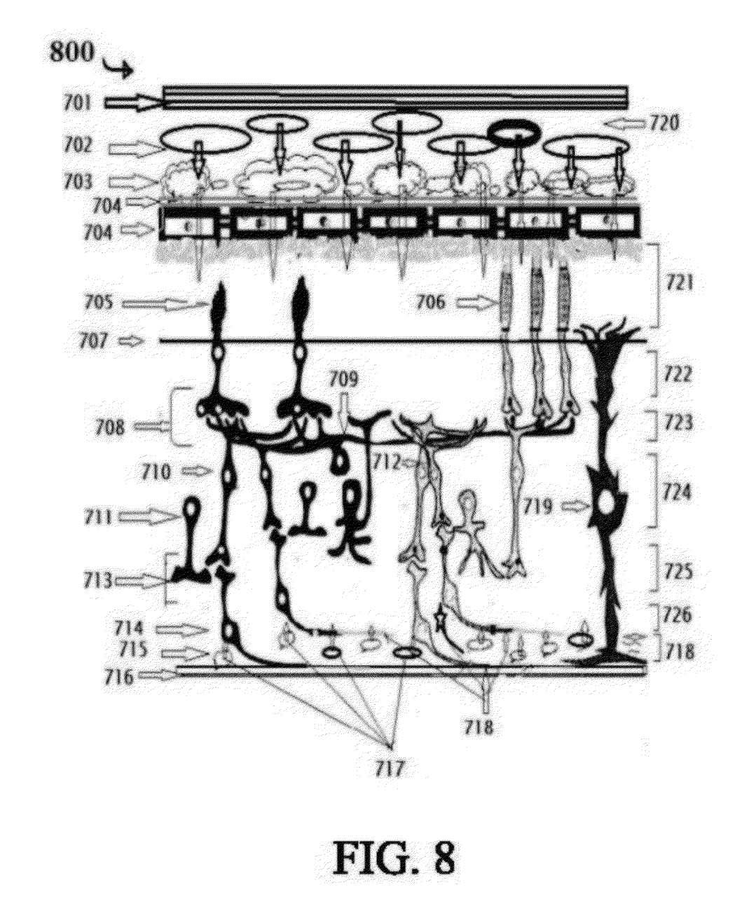

FIG. 8 is a schematic representation 800 showing the histology of the retina in relation to the blood supply and to delineate how the AMD develops and therapeutic agents of this invention reach the site of pathology. This invention of the use of insulin, Monoclonal antibodies and other therapeutic agents reach the rod and cone photoreceptors cells involved in the retinal disease of AMD. It shows sclera 701, large choroidal blood vessels 702, fenestrated choriocapilareis 703 through which the choroidal blood vessels delivers the insulin, Monoclonal antibodies and the other therapeutic agents (indicated by multiple large and the small arrows directed downwards towards rods and cones) of this invention including oxygen and nutriceticals, through the noncelluar Bruch's membrane 704. The Bruch's membrane acts as a interface between the pigment epithelim 704 and choriocappillaries 703 and separates retinal pigment epithelium form the choriocapilaries 703. Due to pathological changes, this membrane becomes 2-3 times thicker in AMD associated with CNV. The cones 705 are not in intimate contact with the retinal pigment epithelium 704. The rods are in close contact with the retinal pigment epthelium brush border 704. The outer limiting membrane 707 formed by the Müller cells 719 separates the photoreceptors outer segments from the rest of the retina in which the separation may prevent the transfer of components from extracellular space of the photoreceptors to the rest of the retina.

In the same fashion, the therapeutic agents get concentrated as they are transported from choriocapillaries towards the outer segment of the photoreceptors, the site of the AMD pathology where this invention is very effective. Note the outer plexiform layer 708, and horizontal cells 709 are the laterally interconnecting neurons in the outer plexiform layer of the retina, and these cells modify and integrate the signals from the rods and cones where the rods and the cones are responsible for allowing eyes to adjust to see equally in bright and dim light conditions. They help to integrate and regulate the input from multiple photoreceptor cells. The bipolar cells 710,712 are situated between photoreceptors (rods 706 and cones 705) and ganglion cells 714.

The therapeutic agents from the conjunctiva do not reach these cells in high concentration due to the presence of outer limiting membrane and absence of vascular network connecting the choroid. The bipolar cells act, directly or indirectly, to transmit signals from the photoreceptors to the ganglion cells. Amacrine cells 711 are the interneurons (40 types are recognized) and they are responsible for 70% of input to retinal ganglion cell 714. The bipolar cells 710, 712 are responsible for the other 30% of input to the retinal ganglian cells. The inner plexiform layer 713, ganglion cell layer 714 receives the signals from the rods and cones through these cells. The inner retinal blood vessels 717 supply oxygen and nutrients to the inner part of retina. They are shown by multiple short arrows pointed towards outer side of the retina. The optic nerve fibers 718 derived from the gangion cells 714 relay the photoreceptors signals to the CNS.

Note the Müller cell 719 contributes to the inner limiting membrane 716 separating the vitreous from the retina and the outer limiting membrane 707. This isolates the sensitive outer segment of the photoreceptors cells of the retina from the rest of the retina. The arrows from choroid indicate the rich vascular supply to the outer segments of the photoreceptors (compared to the rest of the retina), which the outer segments receive the therapeutic agents from the conjunctiva compared to the paucity of BV from the retinal inner BV 717. This diagram shows the insulin, Monoclonal antibodies and other adjuvant therapeutic, pharmaceutical, biochemical and biological agents or compounds from conjunctiva and chorid blood vessels have easy access to rods 706 and cones 705 outer segments in the treatment of AMD.

In one aspect, the trans-conjunctival penetration of insulin and monoclonal antibodies, and therapeutic agents facilitated, by adding the absorption enhancers to the therapeutic agents' composition. The enhancers used to expedite the entry of these agents to penetrate and to permeate inside the eyeball where the agents are delivered to uveal system, and retina. Penetration enhancers may include anionic surfactants, urea, fatty acids, fatty alcohols, terpens, cationic surfactants, nonionic surfactants, Chitin, DMSO, and other such agents.

The inner limiting membrane 716 is the boundary between the retina and the vitreous body. It is formed by astrocytes, the end feet of Müller cell 719 and it is separated from the vitreous humor by a basal lamina. There may be some leaking of aqueous humor from ciliary epithelium and zonule fibers containing insulin, Monoclonal antibodies and other therapeutic agents seeping between these two structures through this basal lamina. This mode of transport or soaking has to be minimal. If it does, the concentration is mostly at mid and anterior part of the lower segment (between 5-7^{o}-clock positions) of the retina due to gravitational drag where the pathology of AMD is prominent (at the mid and anterior part of the retina), but this is not the case in AMD.

It is also possible, that the therapeutic agents from the uveal system (ciliary body, ciliary processes, fenestrated cells of uveal-sclera junction, leak into to vitreous humor also through the Zonular fibers and ciliary body, exerting the therapeutic effect akin to the intra vitreal injection. It is a known fact that the intravitreal injections are performed using monoclonal antibodies or steroids for the treatment of wet AMD. That means that the therapeutic agents transported through the vitreous humour, passing through the inner limiting membrane, various layers of retina, and outer limiting membrane and reach the receptor cell in the macula, neo-choriocapillaries (CNV) that permeates the RPE to have therapeutic effect. Hence, the vitreous humor plays a role in transporting the therapeutic agents from the Conjunctival sac to the site of pathology in AMD across the vitreous.

This diagram 800 also shows various histological layers of the retina. They are as follows: layer of retinal pigment epithelium 704, layer of rods and cones 721, outer nuclear layer 722 made up of nuclei from rods and cones, outer limiting membrane 707 formed by Müller cells, outer plexiform layer 723 made up of synapses between the rods, cones with horizontal and bipolar cells. The inner nuclear layer 724 made up of bipolar and amacrine cell nuclei, inner plexiform layer 725 formed by synapses between the ganglion cells 714, 726, and the process of cells from the inner nuclear layer. The nerve fiber layer formed by the axons of the ganglion cells grouped to become the optic nerve where the nerve fiber leaves the eye at the optic disc to lateral geniculate bodies then to the occipital cortex. The diagram shows how each retinal layer is in touch with the blood vessels; their supply of nurticeuticals, oxygen, insulin, Monoclonal antibodies, and other therapeutic agents used in the treatment of AMD. It is clear that the outer segment of the photoreceptors get the most exposure to the therapeutic agents compared to other functional units of the retina because of their close proximity to the choroid.

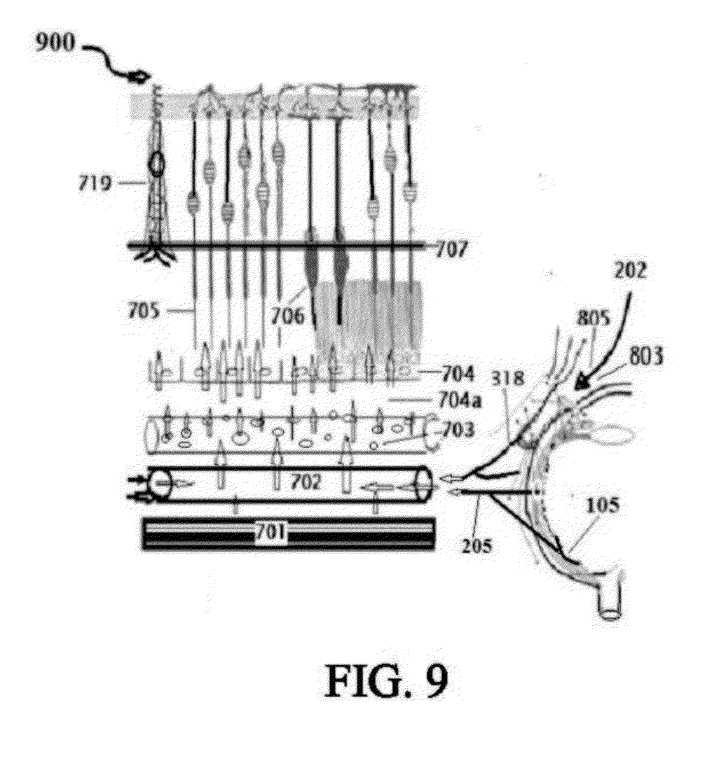

FIG. 9 is a diagrammatic presentation 900 showing the histology of the external layers of retina including photoreceptors. The explanation is the same as FIG. 8. This illustrates the relation to the blood supply to the outer segments of photoreceptors which receives the therapeutic agents delivered through the conjunctional sac. This invention of insulin, monoclonal antibodies and other therapeutic agents reach from the systemic blood supply and conjunctival sac of the eyes to reach the rods and cones photoreceptors cells affected in the pathogenesis of the disease AMD. This diagram shows sclera 701, large choroidal blood vessels 702, fenestrated choriocapillareis 703 deliver the therapeutic agents insulin, Monoclonal antibodies 805, and other therapeutic agents 803 from the ophthalmic drops 202 instilled into conjunctival sac.

The ophthalmic drops 202 of this invention in the conjunctional sac 805 and 803 absorbed by the subconjunctival blood vessels 318, and choroid 205. From here, therapeutic agents delivered to the retina and Macula lutea 105. Insulin and monoclonal antibodies of this invention from the conjunctional sac transported from the choroidal BV 702. then pass to the fenestrated choriocapillares 703 which the choriocapillaries are leaky and the leaked fluid from the inside to extracellular space 707a. This 707a is a cellular Bruch's membrane from this space the Insulin, Monoclonal antibodies passes through the retinal pigment epithelium (RPE) 704 to reach the outer segments of the photoreceptors 705, 706.

The extracellular fluid is bound by RPE and the external limiting membrane 707 formed by the Müller cells 719. The arrows from the choroid indicate the rich vascular supply to the outer segments of the photoreceptors which the photoreceptors receive the therapeutic agents from the conjunctiva. This diagram shows that the therapeutic, pharmaceutical, biochemical and biological agents or compounds from conjunctiva and chorid blood vessels have easy access to rods 706 and cones 705 in the treatment of AMD. The therapeutic agents are transported by the aqueous humor through the suprachoroidal space where the agents permeate to the space between the retinal pigment epithelium and the photoreceptors.

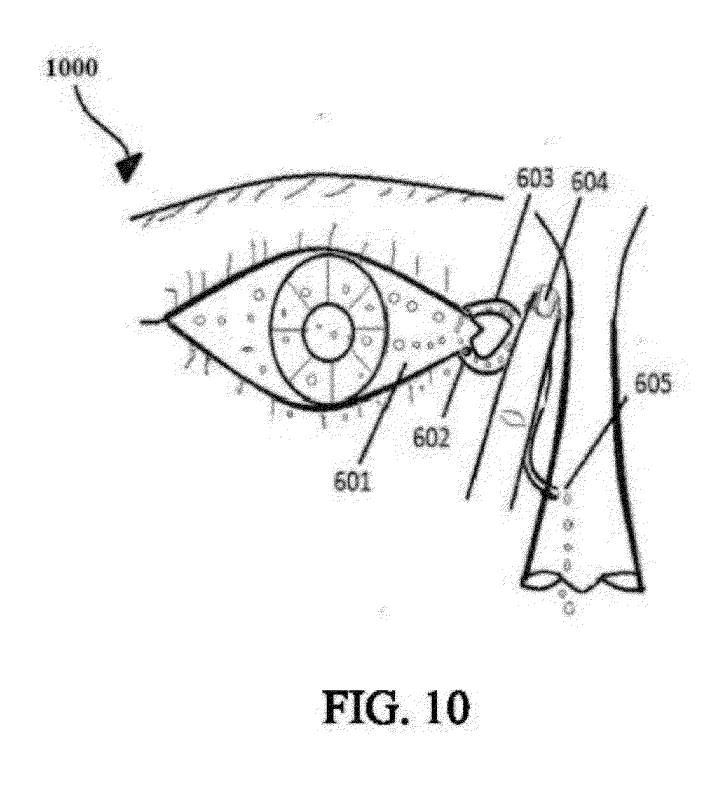

FIG. 10 is a diagrammatic presentation 1000 showing the route of drainage of the lacrimal fluid and therapeutic agents shown as bubbles from the conjunctival fornix (sac) 601 to the nasal mucosa 605 and illustrates a method to prevent the agents from entering the nasal mucosa. A simple method applying the finger pressure 604 at the medial eye angle and nasal junction. The location of the lacrimal punctum, canaliculi 602, 603 and lacrimal sac with a finger 604 will prevent the therapeutic agents drainage to the nasal cavity and the nasal mucosal absorption 605, and their associated systemic adverse effects.

Even now, there is not a single therapeutic agent to cure dry and wet AMD. The etiology of the AMD is still not well established. There are many biological factors implicated in their etiology. The drusen is for sure one of the earliest sign of dry AMD. What changes this into Wet AMD in 10% of the cases still debated? The following discussion may shed some light on the subject. IGF-1 has neurotrophic effect on the neurons in the CNS and probably in the retina, which is nothing but an extension of CNS. That is why it is under investigation for the treatment of ALS. Regrettably, IGF-1 cannot be used if there is diagnosis of wet AMD with CNV formation with or without edema of the RPE and retina. The research studies by Antoinette C Lambooij et al, Showed that the IGF-1 participates in ocular neovascularization, synthesis of IGF-1R and IGF-1—in endothelial cells, RPE cells, and fibroblastic cells, in CNV may point toward a role for this growth factor in the pathogenesis of angiogenesis in neovascular AMD (CNV). (Antoinette C Lambooij et al, Insulin-like Growth Factor-I and its Receptor in Neovascular Age-Related Macular Degeneration. Investigative Ophthalmology & Visual Science. May 2003, Vol. 44, 3, 2192-2198). Vascular endothelial growth factor (VEGF), an endothelium specific mitogen, regarded as one of the most important ocular angiogenic factor, especially under hypoxic circumstances. Other angiogenic factors in ocular neovascularization include basic fibroblast growth factor, transforming growth factor β, platelet derived growth factor, and insulin like growth factor-1 (IGF-1) which are not included in our study.

Research by Rita Rosenthal et al. showed; beside other angiogenic factors like vascular endothelial growth factor (VEGF), insulin-like growth factor (IGF-1 and its receptor, IGF-IR, been implicated in CNV. IGF-I produced in neurons and retinal pigment epithelium (RPE) but its targets and impact in CNV not well understood. IGF-1 Immunoreactivity was rich throughout surgically isolated human CNV tissues and RPE cells were immune-positive for IGF-IR. Cultured RPE cells obtained from CNV tissues expressed IGF-IR. IGF-1 stimulation of cultured cells from CNV tissues induced monophasic sustained rises in intracellular free Ca²⁺ and VEGF concentration in the medium of un-stimulated RPE cell cultures from CNV tissues increased with time to a steady-state (8 h) which was increased two fold by IGF-I stimulation. Thus, in RPE cells IGF-I stimulate the second messenger Ca²⁺ and increases VEGF secretion that, in turn, induces neovascularization (Rita Rosenthal et al. Insulin like growth factor-I contributes to neovascularization in age related macular degeneration. Biochemical and Biophysical Research Communications 323 (2004) 1203-1208). They showed that the RPE cells from eyes without CNV and isolated from CNV tissues respond to IGF-I by secreting VEGF and this effect is likely to be mediated by the second messenger, Ca²⁺, which they demonstrated is increased by IGF-1 in RPE cells. Studies show that the IGF-1 up regulate VEGF expression in RPE cells (R. S. Punglia, M. Lu, J. Hsu, M, Kuroki. M. J. Tolentino, K. Keough, A, P, Levy, N. S. Levy, M. A Goldberg, R. I D'Amato, A. P. Adamis, Regulation of vaseular endothelial growth factor expression by insulin-like growth factorI, Diabetes 46 (1997) 1619-1626). Furthermore, RPE cells are a local source of VEGF (R. N. Frank. R. H. Amin, D, Eliott, J. E. Puklin, G. W, Abrams, Basic fibroblast growth factor, and vascular endothelial growth factor are present in epiretinal and choroidal neovascular membranes, Am. J. Ophthalmol. 122 (1996) 393-403), hence the RPE may be the main culprit in the development of wet AMD.

Etiology of AMD not yet elucidated completely. Nevertheless, investigators have made progress in AMD genetic research by applying genetic epidemiologic methods of analysis. Studies by Haddad et al. showed the possibility of genetic predisposition for this disease (Stephen Haddad, Clara A. Chen, Susan L. Santangelo, and Johanna M. Seddon, The Genetics of Age-Related Macular Degeneration: A Review of Progress to Date. J Sury ophthal 1.51 (4) July-August 2006, Pages 316-363). Their studies suggest how complex the disease is. Their data will help to initiate prophylactic using our method to curb the actions and interactions of multiple genes and environmental factors described in this study.

Studies by Claudio Campa et. al reveal the inflammatory mediators and choroidal neovascularisation (CNV) is the culprit (Claudio Campa et. al. Inflammatory Mediators and Angiogenic Factors in Choroidal Neovascularization: Pathogenetic Interactions and Therapeutic Implications. Mediators of infmmation, Volume 2010, Article ID 546826, pages 14). They describe various processes involved in this CNV. They are inflammatory and endothelial cells factors are key signal in promoting angiogenesis. These include the fibroblast growth factor, transforming growth factor, tumor necrosis factor, interleukins, and complement. It is a known fact that the C-reactive protein and inflammatory cytokine interleukin 6 (IL 6) play a key role in ASVD. This shows the role of inflammatory mediators and angiogenic factors in the development of CNV. It has been shown that in the surgically excised CNV section of patients with CNV, pathologic examination indicates the presence of fragments of Bruch's membrane, RPE, Photoreceptors, vascular endothelium, fibroblasts, macrophages, circulating progenitor/stem cells, and extracellular components including collagen, fibrin, and basal laminar deposits. This point out that the inflammation plays a major role in the development of wet AMD, which may predispose to expression of IGF-1, which in turn stimulates angiogenesis. It is a vicious circle. It is important to note that insulin prevents the adhesion of leukocytes, and removes the ROS, thus help to prevent or lowers the factors including compliments involved in the inflammation responsible for CNV initiation.

Studies by Jha et al show the role of complement system, which control the intraocular inflammation in autoimmune uveitis and play an important role in the development of corneal inflammation, age related macular degeneration (AMD), diabetic retinopathy, and other ocular diseases. Hence, the complement inhibition may have therapeutic application in these ocular diseases (Purushottam Jha, Poran S. Bora, Nalini S. Bora. The role of complement system in ocular diseases including uveitis and macular degeneration Molecular Immunology 44 (2007) 3901-3908). Administration monoclonal antibodies and/or corticosteroids with insulin can lower the inflammatory process in the above-described retinal diseases, preserve the vision, and prevent angiogenesis.

The present invention involves the treatment of etiology, physiology, pathology, signs and symptoms of a variety of eye diseases that grouped under the umbrella of AMD as discussed herein.

One of the important aspects of our invention is the use of insulin in dry AMD, and Insulin with adjuvant therapeutic agent's especially monoclonal antibodies (mAB) in wet AMD. The mAB have therapeutic curative and curtailing effect on wet AMd and prevent the formation of new blood vessels. The use of insulin as prophylactic measures or treatment of the disease in humans or animals described. The method of treatments divided into:
a) Treatment of dry AMD with insulin with other therapeutic measures,
b) Treatment of wet AMD with insulin and monoclonal antibodies,
c) Prophylactic treatment of AMD in the aging population by administering statins (inhibitors of 3-hydroxy-3-methylglutaryl coenzyme A, i.e. HMG-CoA reductase inhibitors); insulin, Luteins, other antioxidants, animal fat free diet, avoiding red meat and adding fish to the meal, and wearing a cool eye mask to reduce the oxidants production (described elsewhere) of metabolism (ROS).

The invention insulin described herein and the effectiveness for treating a variety AMD as:
a) facilitators, carriers, of adjuvant therapeutic agents,
b) To enhance the absorption and to potentiate (augmentation-amplification effects) the effect of therapeutic agents administered to the patients for treatment of AMD and other retinal diseases.
c) to potentiate the adjuvant therapeutic agent action intracellular,
d) the enhance the cell metabolic activity,
e) To promote cell multiplication to replace the apoptotic cells with healthy cells.
f) Conjunctival sac administration of known therapeutic agents, as well as other pharmaceutical, biochemical, nurticeuticals and biological agents or compounds of biologics when compared to systemic administration, carries the following advantages:
g) superior efficacy due to the achievement of higher local concentration at the site of AMD;
h) greater efficacy due to the ability and ease of therapeutic molecule to reach the target tissue without degradation caused by gastrointestinal, hepatic or systemic circulation;
i) more rapid onset of action due to closeness of the therapeutic agents deposition;
j) longer duration of action due to therapeutic agents stasis at the local site;
k) fewer or no systemic side effects, due to lower dosage deposited in the conjunctival sac;
l) ease of administration and greatly improved efficacy due to improved delivery with increased compliance of the therapeutic molecule close to the site of pathology—i.e. Retina;
m) Clinical experience utilizing conjunctival sac route for administration of Adjuvant therapeutic agents with insulin for treating AMD and other oculopathies has demonstrated the dramatic efficacy, and the remarkable rapid onset of action produced by this route of administration. The conjunctional sac already utilized to deliver antiglaucoma therapeutic agents for decades, why not use it to treat AMD?
n) Insulin is “ophthalmologicaly acceptable”.

Diet and AMD Prophylaxis with Insulin Ophthalmic Instillation

Besides various factor blamed as etiological factors in AMD, it becomes clear that food we eat can lower the risk of AMD to 11 times as described below. The inflammation and cholesterol plays an important role in development of AMD that can lead to night blindness described herein. How AMD related to systemic ASVD further supported by the study of what people eat and who develops AMD, and heart disease due elevated cholesterol is reduced by 11 times on low fat diet and taking anti-cholesterol statin adjuvant therapeutic agents. The following studies do support the food we eat and development of AMD with ASVD. The discovery of macular degeneration gene (CPH gene variant is involved in regulating the inflammatory pathways as described) lends support to this hypothesis. Recent research provides additional support. High blood levels of two biomarkers of inflammation—C-reactive protein (CRP) and interleukin 6 (IL-6)—are associated with a twofold increase in the risk of progression of macular degeneration and so also the risk of ASVD. More than 1 serving/week of beef, pork, or lamb as a main dish is associated with a 35% increased risk of macular degeneration as compared with less than 3 servings/month. A high intake of margarine also significantly related to an increased risk of AMD and night blindness. One serving per day of high-fat dairy food (whole milk, ice cream, hard cheese, or butter) increases risk of macular degeneration progression by 1.91 times. 1 serving per day of meat food (hamburger, hot dogs, processed meat, bacon, beef as a sandwich, or beef as a main dish) increases risk of macular degeneration progression by 2.09 times. 1 serving per day of processed baked goods (commercial pie, cake, cookies, and potato chips) increases risk of macular degeneration progression by 2.42 times. People who eat fish more than 4 times/week have a lower risk of macular degeneration than those who consume it less than 3 times/month. People who eat canned tuna more than once per week are 40% less likely to develop macular degeneration as compared with those who consumed it less than once per month. Fish is a major source of DHA (an omega-3 fatty acid). Recently it has been reported that there is a potential beneficial effect of eating any type of nuts on risk of progression of macular degeneration. Eating 1 serving per day of any type of nut reduces the risk of progression of macular degeneration by 40%. This beneficial effect complements other literature reporting a protective role for nuts and cardiovascular disease and type 2 diabetes mellitus. One of the bioactive compounds in nuts, resveratrol, has antioxidant, antithrombotic, and anti-inflammatory properties. We advised all our patients' with any type of AMD, vegetable diet with fish and less red meat and dairy products along with inventive ophthalmic drops described herein. As prophylactic method, all our AMD with ASVD risks with night blindness were on diet rich in fish, vegetable, and nuts with least or no red meat and dairy products at the same time taking statins. Most of these patients reported improved vision, better night vision, lower blood cholesterol, better cardiovascular tolerance with exertion.

Before, the explanation and the description of the disclosed embodiments of the present invention in detail, it be understood that the invention is not limited in its application to the details of the particular examples and arrangements shown. Since the invention is capable of other examples and embodiments in treating other retinal diseases. The terminology used, herein, is for the purpose of description and without a limitation. Earlier enumerated above and narrated below: this application filed in order to disclose Insulin that has high therapeutic activity and metabolism of the photoreceptors cells and RPE. Insulin restores the proper physiological functioning of the retina by acting against the etiological factors such as ROS, genetic defects, correcting any mitochondrial metabolic defect, and restoring the membrane stability. It enhances the effectiveness (augmentation-amplification effects) of other adjuvant therapeutic, pharmaceutical, biochemical, and biological agents or compounds used in the treatment of age related macular degeneration and other retinal diseases. Insulin, of the present invention, helps to maintain functional and structural integrity of the photoreceptors when they have genetic defects. Furthermore, this invention insulin helps to delay the expression of genetic defects that there is genetic defects exist in the photoreceptors by mopping the ROS, which these genetic defects predisposes or causes the age related macular degeneration.

At present, the insulin exclusively used to treat type I and certain cases of type II diabetes. Our discoveries and inventions describes the use topically (locally) in other disease conditions besides diabetes that includes: cancers, dry eye syndrome, glaucoma, prostate diseases, middle and inner ear afflictions, age related changes of the facial skin, healing of wounds, gum diseases; to treat hair loss, enhancing eye lashes and alleviated local infections. Insulin use systemically or locally also includes CNS diseases including autism, Parkinson's disease, depression, Alzheimer's, obesity; for activating vaccines, cytokines, Lymphokine, monoclonal antibodies, activating local immune system at lymph nodes; enhancing the local effects of chemotherapeutic agents; in treatment of autoimmune diseases to enhance the activity of monoclonal bodies, and multiple local and systemic therapeutic applications.

Insulin, and its Biological Effects on and the Role Plays in the Uptake, Distribution; Augmentation-Amplification Effects of Other Adjuvant Therapeutic Agents Used in the Present Invention to Treat AMD.

A variety of carriers, adjuvant agents, absorption enhancers, potentiators (augmentation/amplification effects) of therapeutic agents, cell metabolic activity enhancers, cell multiplication enhancers (mitotic), and other methods have been used to enhance the absorption and to potentiate the effect of therapeutic agents. They augment and amplify the effects pharmaceutical, biochemical, and biological agents or compounds administered to the patients for improving the physiological function, and for the treatment of diseases. Such endocrine biological agent is Insulin, used of this invention.

It known that the Insulin benefits the post ischemic myocardium by stimulating pyruvate dehydrogenase activity. This activity in turn stimulates aerobic metabolism of cardiac and other tissue reperfused. Insulin increases the glutathione synthesis by activating gamma-glutamyl-cysteine synthetase, which is a powerful antioxidant. This physiological effect can have impact on repairing and restoring the photoreceptors and RPE after the onslaught of ROS and prevent or curtail the development of ARD. Insulin increased redox status by increasing intracellular glutathione (GSH) content in oxidized cells. This reduced the ROS from the cells will cure, and curtail retinal diseases including AMD, by mopping the ROS. The insulin metabolic affects reduces both polymorphonuclear neutrophils adhesion due to ROS (reactive oxygen species—ROS—free radicals). This effect can reduce the inflammatory processes involved in the CNV angiogenesis. Insulin augments the DNA, RNA, and protein synthesis that results in increased growth by mitosis (Osborne C K, et al. Hormone responsive human breast cancer in long-term tissue culture: effect of insulin. Proc Natl Acad Sci USA. 1976; 73: 4536-4540). It enhances the permeability of cell membranes to many adjuvant therapeutic agents including antiangiogenic monoclonal bodies, neurotrophic agents, and antioxidants. Besides glucose, and electrolytes; Insulin helps and facilitates to move the drugs and therapeutic agent molecules from extra cellular fluid (ECF) to intracellular fluid (ICE) meaning from outside the cells to inside the cells thus facilitates the uptake of therapeutic agents in the treatment of AMD.

Insulin has properties of tissue growth factors, and regulates growth and energy metabolism at the whole organism level farther away from the site of production and application in the conjunctival sac. This is the reason the use of Insulin with or without adjuvant therapeutic agents topically not only has the local effect; they are absorbed and circulated farther away from the site of application (endocrine effect) and exert their therapeutic effects on the rods, cones, RPE, Muller cells and other neuronal complex in the retina.

Insulin will exert endocrine, paracrine, intracrine effect (Hernandez-Sanchez C, Lopez-Carranza A, Alarcon C, de la Rosa E J. de Pablo F. Autocrine/paracrine role of insulin-related growth actors in neurogenesis: local expression and effects on cell proliferation and differentiation in retina. Proc Natl Acad Sci USA. 1995; 92:9834-9838.), and enhance the absorption, and action of monoclonal anti-angiogenic antibodies, antioxidants, and other such therapeutic agents inside the choroidal BV, Burch's membrane, RPE, photoreceptors and Muller cells by maintaining the health these eye structures which otherwise contribute to AMD. Once inside the choroid-retinal complex, the insulin augments and amplifies the effects of adjuvant therapeutic agents (intracrines effects) and any adjuvant agent proven and approved to treat AMD such as monoclonal antibodies by restoring their physiological function (Alabastor IBID). The results show that glutathione (GSH) generation with the help of the insulin can reverse the effect of oxidative damage (oxidative free radical damage-ROS) by tyrosine kinase activation and phosphorylation.

In an ingenious vitro studies, this effect of augmentation and amplification effects of insulin shown, in that the insulin activates and modifies metabolic pathways in MCF-7 human breast cancer cells by paracrine, and intracrines effects. The insulin increases the cytotoxic effect of methotrexate up to 10,000 (ten thousand times-augmentation and amplification effects) folds (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228). Our studies supports the findings of Alabastor (IBID) that the disease or the healthy cell sensitivity to the therapeutic and biological agents as those to be used to enhance night vision and treat AMD in the presence of insulin (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January 2009, Issue #306, pages—106-110).

The retina is nothing but an extension of the brain; hence, the effect of these therapeutic agents on the Burch's membrane, RPE, photoreceptors, retina and Muller cells is similar to the effects on the CNS. Therefore, insulin play an important role in maintaining proper integrity, growth, repair, regeneration, moping the ROS, mitochondrial health, and functioning of the eye's choroid, Burch's membrane, RPE, photoreceptors and Muller cells in particular.

The insulin induces cell growth, mitosis, enhances metabolism, increases the glutathione synthesis needed for health (besides glucose transport) of the photoreceptors. This enhanced mitosis, increases the production of nuclear proteins in the nucleus and ribonucleoprotein production by the endoplasmic reticulum, activates the Golgi complex; enhances the lysosomes activity. Thus, the insulin and helps to break up endocytosed toxic substances, cellular debris, and to eliminate the cellular toxins within the photoreceptors cells (augmentation/amplification effects). The insulin, deposited in the conjunctival sac, will enhance the uptake of antioxidants and other adjuvant therapeutic, pharmaceutical, biochemical and biological agents or compounds by the dysfunctional cells of the retina. They mop up the ROS to prevent further damage to the rods and cones and to restore the function of the retina in AMD described in this inventive method (Shantha, T. R. Site Of Entry Of Rabies Virus Form The Nose And Oral Cavity; And New Method Of Treatment Using Olfactory Mucosa And By Breaking BBB, presented at The 2nd International Rabies In Asia Conference Held In Hanoi, 2009, Pp 70-73, and The Rabies in the North Americus (XX RITA), held in Quebec City, 2009, Pp 20-21, Rabies Cure: United States Patent Application Publication No.: US 201110020279 A I, Rabies cure, Totada R. Shantha).

It is important to emphasize that the use of insulin ophthalmic drops of this invention after intravitreal injection of stem cells can enhance their mitosis, seeding, and facilitate regeneration of RPE and photoreceptors afflicted in AMD as well as in retinitis pigmentosa.

Thus, the present inventive method not only enhances the uptake of adjuvant therapeutic agents, but also enhances their therapeutic effect inside the photoreceptors afflicted cells as reported by Alabaster (IBID). The IGF-1 has potential angiogenesis effect; hence, we do not use this biological agent in wet AMD and other retinal diseases associated with angiogenesis. On the other hand, it may be effective in the treatment very early stages of dry AMD with insulin and other adjuvant therapeutic agents to maintain the integrity of photoreceptors, because it is neurotrophic factor. We have used it, in our practice, in small doses with insulin without any angiogenesis effects.

In one aspect, the trans-conjunctival penetration of insulin and adjuvant therapeutic agents facilitated, by adding the absorption enhancers to the therapeutic agents' composition. The enhancers used to expedite the entry of these agents to penetrate and to permeate inside the eyeball where the agents delivered to uveal system, choroid, and macula lutea of the retina. Penetration enhancers may include anionic surfactants, urea, fatty acids, fatty alcohols, terpens, cationic surfactants, nonionic surfactants, Chitin, DMSO, and other such agents.

There are various forms of insulin used to treat diabetes. Insulin products classified according to their putative action as rapid, short, intermediate, and long acting insulin. We have used rapid acting, short acting, and long acting protamine zinc insulin in our studies. Protamine Zinc Insulin is long acting insulin contains Zinc. Zinc is an antioxidant; hence, this form of insulin is even more effective in reducing the effect of ROS. Because of its zinc content, it is included in compounding of the ophthalmic drops in this invention.

The dose of insulin is 0.5, 1 to 2 IU per eye per drop. The dose be decreased or increased depending upon the age, weight, and severity of the AMD affliction in a given patient.

There is a possibility of developing hypoglycemia when the insulin used as indicated by signs and symptoms such as rapid heartbeat, sweating, dizziness, confusion, unexplained fatigue, shakiness, hunger, feeling hot, difficulty in thinking, confusion. Such patients should be treated with oral ingestion of a fast-acting carbohydrate such as glucose tablets, fruit juice, fruit bowl, chocolate bar, regular Coca-Cola, sugary drinks or eat plain sugar followed with a drink of water or IV administration of 25% glucose, if the reaction is severe.

Any treatment of age related macular degeneration with or without other retinal diseases with ophthalmic topical preparations (eye drops) designed in our invention using Insulin in dry and wet AMD with other adjuvant therapeutic agents as prophylactic, and/or for treatment encompass the following principles:
a) Eye drops, semi liquids, gels or ointments should act like a film covering like natural tears over the ocular surface of the eye including cornea with less stinging or burning sensation,
b) The above are capable of providing mechanical lubrication for the ocular surface, which the eyelid glides easily during the blinking movement.
c) The reduction of the evaporating natural lacrimal fluid,
d) The emulsion or the watery ophthalmic drops shouldn't react with eye cellular structures, the lacrimal coating, and the eye lid lacrimal glandular system and opthalmologicaly acceptable.
e) Eye drops should be stable for a reasonable period at room temperature.
f) The therapeutic preparations should be easily absorbed with or without other absorption enhancers if possible and transported to the site of the pathology.
g) Besides acting against age related macular degeneration pathology, the therapeutic preparations should contain therapeutic, pharmaceutical, biochemical and biological agents or compounds capable of alleviating the underlying cause responsible for other oculopatheis including AMD; at the same time augments and amplifies the effects of therapeutic agents with trophic effects when used with our invention.
h) The ophthalmic therapeutic agents should have therapeutic healing effects on other oculopathies, which are specific for retinal diseases, that it is used.
i) In our invention, insulin based ophthalmic preparations meet all the above-recited physiological, pharmacological, and therapeutic parameters.
j) Additional adjuvant agents included in the ophthalmic compounding to preserve the solution, maintain proper photoreceptors, facilitate the uptake of the therapeutic agents, protect the eyes, and at the same time have therapeutic effect on other oculopathies.

Insulin and adjuvant therapeutic agents are compounded as a liquid ophthalmic isotonic solution other antiautoimmune therapy agents (monoclonal antibodies), or vitamins, and one or more one buffering agents, said buffering agents producing a pH in said composition similar to mammalian eye fluids.

Dosing with respect to the amount of bioactive agent such as insulin is dependent on the type, severity, and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. For example, insulin may be used a bioactive agent, and the insulin can be short or long acting and dosing may include 3, 5, 10, 15, 20, 30, 40, International Units per milliliter delivery system as ophthalmic drops.

Oxidizing the reduced glutathione to prevent breaking of disulphide bond of insulin, absorption enhancers, wetting agents, lubricant, solvents, and other therapeutic agents in preparing the ophthalmic drops:

As described herein, the pharmaceutically acceptable oxidizing agent facilitates the delivery of the bioactive agent through the mucosal membrane. In general, the oxidizing agent can react with molecules present in the conjunctional sac mucosal membrane that would adversely react with the bioactive agent. For example, reduced glutathione can inactivate bioactive agents by breaking crucial molecular bonds. Not wishing to be bound by theory, when delivering insulin either transmucosally or transdermally, reduced glutathione can inactivate insulin. Specifically, insulin has numerous disulfide bonds, which are crucial for its protein conformation, biological activity, and subsequent therapeutic effects. Reduced glutathione will inactivate insulin by reducing or breaking insulin's disulfide bonds. Once these disulfide bonds are broken, insulin becomes inactive due to lost protein conformation and biological activity. Thus, the administration of the oxidant or oxidizing agents using the devices described herein prevents the inactivation of the bioactive agent. Specifically, applying an oxidant or a pharmaceutically oxidizing agent transmucosally will lower or prevent the effects reduced proteins and reduced biological molecules have on the bioactive agents. In this manner, the inactivation of bioactive agents via reduction or cleavage of crucial molecular bonds avoided. The selection and amount of the pharmaceutically acceptable oxidizing agent can vary depending upon the bioactive agent that is to be administered. In one aspect, the oxidizing agent includes, but is not limited to, iodine, povidone-iodine, any source of iodine or combinations of oxidants, silver protein, active oxygen, potassium permanganate, hydrogen peroxide, sulfonamides, dimethyl sulfoxide or any combination thereof. These oxidizing agents may also act as absorption agents which help facilitate delivery of a therapeutic agent onto and into a mucosal membrane. In one aspect, the oxidant is at least greater than 1% weight per volume, weight per weight, or mole percent. In another aspect, the skin permeability enhancer may be at least greater than 1.5%, 2.0%, 2.5%, 3.0%, 3.5%, 4.0%, or 4.5% weight per volume, weight per weight, or mole percent. In this aspect, the oxidant may range from 2% to 10%, 2% to 9.5%, 3% to 8%, 3% to 7%, or 4% to 6% weight per volume, weight per weight, or by mole percent.

Interestingly, the conjunctional lining has very thins layer of strum corneum and hence hardly any glutathione to inactivate the insulin. We have used povidone iodine in our studies as an oxidizing agent of glutathione. We use 0.1% to 0.05% povidone iodine (PVP-I) solution in normal saline. It can be mixed with 40 IU of insulin per milliliter, so that it can be delivered to the conjunctival sac as drops with insulin. 2.5% buffered PVP-I solution is already in use for prophylaxis of neonatal conjunctivitis (Ophthalmia neonatorum) which can lead to blindness, especially if it is caused by Neisseria gonorrhoeae, or Chlamydia trachomatis. PVP-I is suitable for this purpose because unlike other substances it is efficient also against fungi and viruses (including HIV and Herpes simplex). It is proved harmless to ocular structures in the newborn so also in adults.

Additional components can be present in the ophthalmic solution to facilitate the delivery of the bioactive agent mucosally to the subject. In one aspect, transmucosal penetration enhancers can be used to further expedite the entry of the bioactive agent into the mucosa and ultimately the blood stream. Penetration enhancers work by increasing permeability across a particular boundary or membrane. Penetration enhancers not only penetrate a membrane efficiently, but these enhancers also enable other bioactive agents to cross a particular membrane more efficiently. Penetration enhancers produce their effect by various modalities such as disrupting the cellular layers of mucosa, interacting with intracellular proteins and lipids, or improving partitioning of bioactive agents as they come into contact with the mucosal membranes. With these enhancers, macromolecules up to 10 kDa are able to pass through the mucosal membrane.

These enhancers should be non-toxic, pharmacologically inert, non-allergic substances. In general, these enhancers may include anionic surfactants, ureas, fatty acids, fatty alcohols, terpenes, cationic surfactants, nonionic surfactants, zwitterionic surfactants, polyols, amides, lactam, acetone, alcohols, and sugars. In one aspect, the penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate, dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethyl-hexyl pelargonate, butyl benzoate, benzyl benzoate, benzyl salicylate, dibutyl phthalate, or any combination thereof. In one aspect, the skin permeability enhancer is at least greater than 1% weight per volume, weight per weight, or mole percent. In another aspect, the skin permeability enhancer may be at least greater than 1.5%, 2.0%, 2.5%, 3.0%, 3.5%, 4.0%, 4.5% up to 50% weight per volume, weight per weight, or mole percent. In one aspect, the skin permeability enhancer is dimethyl sulfoxide. In this aspect, the amount of dimethyl sulfoxide may range from 2% to 10%, 2% to 9.5%, 3% to 8%, 3% to 7%, or 4% to 6% weight per volume, weight per weight, by mole percent, or any effective therapeutic amount.

In other aspects, these additional components may include antiseptics, antibiotics, anti-virals, anti-fungals, anti-inflammatories, anti-dolorosa, antihistamines, steroids, and vasoconstrictors within the device to reduce inflammation or irritation on and around the mucosal membrane. Such vasoconstrictors may include phenylephrine, ephedrine sulfate, epinephrine, naphazoline, neosynephrine, vasoxyl, oxymetazoline, or any combination thereof. Such anti-inflammatories may include non-steroidal anti-inflammatory drugs (NSAIDs). NSAIDs alleviate pain and inflammation by counteracting cyclooxygenase and preventing the synthesis of prostaglandins. In one aspect, NSAIDs include celecoxib, meloxicam, nabumetone, piroxicam, naproxen, oxaprozin, rofecoxib, sulindac, ketoprofen, valdecoxid, anti-tumor necrosis factors, anti-cytokines, anti-inflammatory pain causing bradykinins or any combination thereof. Such antiseptics, anti-virals, anti-fungals, and antibiotics, may include ethanol, propanol, isopropanol, or any combination thereof; a quaternary ammonium compounds including, but not limited to, benzalkonium chloride, cetyl trimethylammonium bromide, cetylpyridinium chloride, benzethonium chloride, or any combination thereof; boric acid; chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof.

Adjuvant therapeutic biological agents for the treatment of dry AMD and wet AMD with insulin ophthalmic conjunctional sac instillation

Ranibizumab, (LUCENTIS™) is a type of monoclonal antibody target a particular protein and locks with it, affecting its function. It is called targeted therapy. A monoclonal antibody is a man-made version of an immune system protein that fits like a lock and key and attaches to a vascular endotelial growth factor (VEGF) protein, which is required to grow new blood vessels (BV) as seen in wet AMD. Ranibizumab, a Fab (fragment antibody binding) fragment derived from the same parent molecule as bevacizumab (Avastin™), also developed by Genentech (by the same scientist Napoleone Ferrara) for intraocular use and is FDA approved for ophthalmic use to treat wet AMD. It has undergone extensive clinical trials. Reports indicate substantially better outcomes in patients treated with intravitreal Ranibizumab than conventional treatments in people with choroidal neovascularization (CNV—wet age related macular degeneration—wet AMD). Most patients with choroidal neovascularization lose vision or at best maintain vision despite treatment with laser, photodynamic therapy, or Macugen. A much larger proportion (up to 70%) gained vision with Ranibizumab.

Bevacizumab, from which Ranibizumab is developed, referred to as an anti-angiogenic drug. It stops tumors from being able to create new blood vessels to feed the tumor, supply of nutrients, which in turn slow or stop their growth and metastasis. In the same fashion, Ranibizumab and Bevacizumab curtails or stops the new development BV in wet AMD, shriks vasucalr mass, and reduces inflammatory stimulation of angiogenisis and reduces or eliminate retinal edema. By this machinism, it inhibits the edema and damage to the underlying RPE and Photoreceptors of the macula and the rest of the retina. It also prevent the new CNV vessels formation and make the existing chorio-capillaries more stable, and allowing the other therapeutic agents be more effective locally and exert their effect at the site of wet AMD.

The disadvantage of these antiangiogenic agents is that they are to be injected intravitrealy every 6-8 weeks in doses of 0.3 to 1.2 mg in 0.01 ml using 30 gauge needle. Our invention of using it insulin with Bevacizumab and Ranibizumab obviates intravitreal injection and makes patients more complaint with the treatment modality. One of the drawbacks of Ranibizumab is financial; it is 50 times more expensive than Bevacizumab, which has similar effect. Claims made that it is 2.5 times more effective than a similar drug Bevacizumab contrary to the published studies.

The marketers claim that the Ranibizumab is a smaller molecules compared to Bevacizumab, which is thought to give Ranibizumab an advantage over Bevacizumab in its ability to penetrate the eye's retina and halt abnormal blood vessel growth contributing to advanced macular degeneration and scarring that causes blindness.

Bevacizumab (trade name AVASTIN™, Genentech/Roche) is a drug that blocks angiogenesis, the growth of new blood vessels. We have used this monoclonal antibody in the treatment of advanced cancers in very large doses. Bevacizumab is a humanized monoclonal antibody that inhibits vascular endothelial growth factor A (VEGF-A). VEGF-A is a chemical signal that stimulates angiogenesis in a variety of diseases, especially in cancer and in wet AMD. Bevacizumab was the first clinically available angiogenesis inhibitor in the United States. It is used in the treatment of various cancers, including colorectal, lung, breast, kidney, and brain (glioblastomas).

Many diseases of the eye, such as age-related macular degeneration (AMD) and diabetic retinopathy, damage the retina and cause blindness when blood vessels around the retina grow abnormally and leak fluid, causing the layers of the retina to separate. This abnormal growth caused by VEGF, so bevacizumab successfully used to inhibit VEGF and slow this growth of BV in wet AMD.

Recently, Bevacizumab been used by ophthalmologists as an intravitreal injection agent in the treatment of proliferative (neovascular) eye diseases, particularly for choroidal neovascular growth (CNV) in AMD. Although not currently approved by the FDA for such use, the injection of 1.25-2.5 mg of bevacizumab into the vitreous humor performed without significant intraocular adverse effects and toxicity. There are hardly any systemic toxicity, because the dose is minimal compared to its use for cancers. Many retina specialists have noted impressive results in the setting of CNV, proliferative diabetic retinopathy, neovascular glaucoma, diabetic macular edema, retinopathy of prematurity and macular edema secondary to retinal vein occlusions.

When bevacizumab used in the treatment of macular degeneration, only tiny and relatively inexpensive doses (compared to amounts used in colon and other cancers) are required. Some investigators believe that bevacizumab at a cost of around $42 a dose is as effective as Ranibizumab at a cost of over $1,593 a dose (approximately).

Bevacizumab (100 mg/4 ml) Solution is a monoclonal antibody used to treat certain types of advanced lung cancer, certain types of brain, breast, kidney, colon, or rectal cancers with other anti-cancer therapies. Bevacizumab 1.25 mg intavitreous injections at six weeks interval given as part of a six weekly variable retreatment regimen is superior to standard care (pegaptanib sodium, verteporfin, sham), with low rates of serious ocular adverse events. Treatment improved visual acuity on average at 54 weeks. It is important to note that the effectiveness of these monoclonal antibodies enhanced by the use of insulin drops after intravitreal injection.

Bevacizumab versus Ranibizumab effectiveness: The National Eye Institute (NEI) of the National Institutes of Health (NIH) announced in October 2006 that it would fund a comparative study trial of ranibizumab (Lucentis®) and bevacizumab (Avastin®) to assess the relative safety and effectiveness in treating AMD. This study, called the Comparison of Age-Related Macular Degeneration Treatment Trials (CATT Study), enrolled about 1,200 patients with newly diagnosed wet AMD, randomly assigning the patients to one of four treatment groups. Results of the study released on Apr. 29, 2011. The study found that the benefits of both Bevacizumab and ranibizumab are essentially identical after one year. This has a significant impact because the price difference between the two medications means insurance providers and Medicare will fund treatment with Bevacizumab in preference to the higher priced ranibizumab-Lucentis. It was reported, from the Comparison of AMD Treatments Trials (CATT), was published online in the New England Journal of Medicine on Sunday, May 1, 2011.

Pegaptanib (MACUGEN™): Pegaptanib is a pegylated anti-VEGF aptamer, a single strand of nucleic acid. It binds with specificity to VEGF 165, a protein that plays a critical role in angiogenesis (the formation of new blood vessels) and increased permeability (leakage from blood vessels-causing macula lutea edema), two of the primary pathological processes responsible for the vision loss associated with neovascular AMD. The FDA approved it to treat wet macular degeneration in December 2004. Macugen is injected into the eye every six weeks, in 0.3 mg doses at a time six weekly according to the American Macular Degeneration Foundation website. Macugen slows down visual loss from wet macular degeneration. Pegaptanib decreases the level of a protein that affects the cells of the eye. This protein can cause swelling and blood vessel changes that lead to macular degeneration and blindness.

Triamcinolone acetonide (KENALOG™) and other corticosteroids: Corticosteroid decrease inflammation and stabilizing the membranes of the intracellular organelle, a known physiological function. The researchers and the ophthalmologists have been evaluating the use of the corticosteroid, Kenalog® in treating wet macular degeneration for several years. This therapeutic agent injected into the vitreous in the back of the eye. One study by M. C. Gilles and colleagues published in a 2003 issue of “Archives of Ophthalmology” found that the medication had no effect on the risk of vision loss when compared to no treatment at all. Another study by J. B. Jonas and colleagues in a 2004 issue of the “Archives of Ophthalmology” found that multiple injections improve visual acuity in patients with wet macular degeneration. Adequate studies to demonstrate the safety of Triamcinolone acetonide Injection use by intra-turbinal, subconjunctival, sub-Tenons, retro-bulbar, and intraocular (intravitreal) injections not been performed.

Triamcinolone acetonide not approved by the FDA, but research for AMD treatment is ongoing. Corticosteroids not used in active ocular herpes simplex. Many of these complications are common to most the therapeutic agents delivered intravitreal. These effects are almost non-existent using monoclonal antibodies and corticosteroids with insulin as ophthalmic drops instilled into Conjunctional sac in our studies instead of intravitreal injection.

For the purpose of comparison, the following is the equivalent milligram dosage of the various glucocorticoids:

| | |
|---|---|
| Cortisone, 25 | Triamcinolone, 4 |
| Hydrocortisone, 20 | Paramethasone, 2 |
| Prednisolone, 5 | Betamethasone, 0.75 |
| Prednisone, 5 | Dexamethasone, 0.75 |
| Methylprednisolone, 4 | |

Triamcinolone acetonide (KENALOG®) injections been used to treat the following eye diseases. They are pseudophakic cystoid macular edema that fails to respond to conventional therapy; clinically significant diffuse diabetic macular edema that fails to respond to conventional laser treatment; macular edema associated with branch retinal vein occlusion that fails to respond to laser treatment (or where laser has not been shown to be useful); non-ischemic central retinal vein occlusions associated with decreased vision with or without macular edema; and Select cases of wet AMD, often in combination with photodynamic therapy with verteporfin (VISUDYNE).

Usually 0.3 cc (13.13 mg) of KENALOG™ (40 milligrams per milliliter) is injected intra-vitreal using a thin gauge needle. We have used dexamethasone (DECADRON™) with insulin in our treatment of AMD and other retinal diseases mentioned above ophthalmic drops with insulin instead of intravitreal injection and avoid this invasive procedure.

Side effects and Complications of intravitreal injection avoided using these therapeutic agents with insulin instilled into conjunctional sac: Following are some of the ocular complications of intra vitreal injection. After the intravitreal injection, the patient may notice slight blurriness and swirls in the vision for a few days, redness, bloody eye, and irritation, and increased watering of the eye, settle after a few days. Pain, Intravitreal bleeding, Endophtalmitis, glaucoma after intravitreal injection of Bevacizumab and ranibizumab are some of the important complications though rare need to bear in mind. The adverse side effects of KENALOG® include cataract formation, secondary ocular infections due to bacteria, fungi, or viruses, and rarely endophthalmitis, retinal detachment, hemorrhage, posterior sub capsular cataracts, glaucoma with possible damage to the optic nerves, and visual disturbances including vision loss been reported with intravitreal administration. We prevent using corticosteroids in the presence of active eye infections. Using ophthalmic drops of insulin with monoclonal antibodies or other therapeutic agents described in this invention instead of intravitreal injection preclude these serious complications.

Insulin ophthalmic drops with nutraceutical supplement for the treatment of AMD

A large research study from Harvard showed that supplementing with 6 mg of lutein per day orally could reduce likelihood of getting macular degeneration by 57% (Seddon, J. M., U. A. Ajani, et al. (1994). “Dietary carotenoid, vitamins A, C, E, and advanced age-related macular degeneration. Eye Disease Case-Control Study Group JAMA, 272(18):1413-20). Same study showed that the specific carotenoids, lutein and zeaxanthin, which are primarily obtained from dark green leafy vegetables, were most strongly associated with a reduced risk for AMD. Individuals consuming the highest levels of carotenoids had a statistically significant 43% lower risk for AMD. The AMD study showed that supplementing with a combination of beta-carotene, vitamins C and E, zinc and copper could significantly reduce the chances of dry macular degeneration turning to wet macular degeneration. Therefore, there certainly are preventative measures you can take. Additional beneficial nutrients include omega-3 fatty acids, taurine, vitamins A and E, selenium, zinc copper, beta-carotene, gingko biloba. For those with macular degeneration, research has shown that this is a condition that can be very responsive to specific nutritional supplementation (lutein, zeaxanthin, taurine, omega-3 fatty acids, vitamins A and E, selenium, beta-carotene, zinc and copper to name a few), diet and lifestyle. After the nutriceutical intake, wait an hour and then administer insulin to facilitate their uptake and augment their effectiveness as they reach the retinal circulation.

HMG-Co A reductase inhibitors against AMD development: According to Catharine Gale et al, U.S. Patent Application Publication Number: 2003/0065020), in a cross-sectional survey of men and women who use statins is associated with an 11-fold reduction in risk of macular degeneration. This tells us that the hypercholesterimia connected to the production of drusen. Statins are inhibitors of 3-hydroxy-3-methylglutaryl coenzyme A, i.e. HMG-CoA reductase inhibitors. Accordingly, we provide that age-related macular degeneration (AMD) is effectively treated by administration of HMG-CoA reductase inhibitors such as statins. Furthermore, administrations of such HMG-CoA reductase inhibitors are effective in preventing the occurrence of age-related macular degeneration. Despite laser treatment, the disease and loss of vision may progress, and once vision is lost, it cannot be return. No specific medical treatment is currently available for macular degeneration to cure or curtail. Hence we have taken measures to put all patients' with elevated cholesterol on statins selected from the group consisting of: fluvastatin (LESCOL), cerivastatin (BAYCOL), atorvastatin (LIPITOR®), simvastatin (ZOCOR®), pravastatin (PRAVACHOL), lovastatin (MEVACOR®) and rosuvastatin (ZD 4522) therapeutic agents with insulin ophthalmic drops as prophylactic to prevent or curtail AMD development in the future. We used atorvastatin (LIPITOR®) to reduce the cholesterol. These patients put on regimen of low cholesterol, red meat and dairy product free, vegetable plus fish diet. This provides a method for (a) lowering the level of LDL cholesterol; (b) increasing the level of HDL cholesterol; and (c) lowering the level of triglycerides in the patient. Hence, prevent or inhibit the growth of drusen, important culprit in AMD related blindness. This will also prevent the development of the AMD in the second eye if the first eye diagnosed with AMD, with one normal eye.

Chelation: Ethylenediaminetetraacetic acid (EDTA) is used extensively in the analysis of blood. It is an anticoagulant for blood samples for CBC/FBEs. Laboratory studies also suggest that EDTA chelation may prevent collection of platelets on the lining of the vessel [such as arteries] (which can otherwise lead to formation of blood clots, which itself is associated with atheromatous plaque formation or rupture, and thereby ultimately disrupts blood flow). EDTA is highly effective in reducing bacterial growth during implantation of intraocular lenses (IOLs). Several theories suggested by doctors who recommend this treatment of EDTA for coronary heart disease. One theory suggests that EDTA chelation work by directly removing calcium (as well as lead, copper, iron) found in atheroma plaques that block the arteries, causing them to break up, that in turn causes calcium to be removed from the plaques or causes a lowering of cholesterol levels. It also works by reducing the damaging effects of oxygen ions (oxidative stress) on the walls of the blood vessels, which could reduce inflammation in the arteries and improve blood vessel function. Hence, it is an ideal ophthalmic drop to prevent AMD and development of wet AMD. With insulin, the effect of EDTA is augmented and amplified many times. As prophylactic and in early cases of AMD, the 5% compounded EDTA provided to the patients. After prolonged use of 3 months, the patient did report improvements in vision.

It is a known fact that the photoreceptors in AMD and age related macular degeneration are undergoing changes and apoptosis due to deposits of fat, calcium, protenacious, and dysfunctional cellular complexes including iron from the choriocapillaries. These changes take place in the choroid, RPE, Bruch's membrane, photoreceptors, and Muller cells. Using insulin drops with EDTA, a well-known chelation agent can soften the drusen and help to remove them, as seen in ASVD of the coronary blood vessels. I do believe that the drusen are akin to atherosclerotic patches in the BV.

Glutamate toxicity and AMD: Glutamine (Gln), glutamate (Glu) and γ-amino butyric acid (GABA) are essential amino acids for brain and retinal metabolism and function. Astrocytic-derived (in the eyes Muller cells) glutamine is the precursor of the two most important neurotransmitters: glutamate, an excitatory neurotransmitter, and GABA, an inhibitory neurotransmitter. Glutamine is a derivative of glutamic acid. Its chemical name is glutamic acid 5-amide.

Reactive oxygen species with liberation of glutamate are produced due to photon induced light perceptions and hypoxia (due to drusen deposits) results in dysregulation of RPE and photoreceptors metabolism. It is a known fact that glutamate plays a major role in excitotoxicity of CNS and retina. Research shows that glutamate receptors are present in CNS glial cells as well as neurons, so also retina including RPE (Steinhäuser C, Gallo V (August 1996). “News on glutamate receptors in glial cells”. Trends Neurosci. 19 (8): 339-45) and in Muller cells of the retina. The glutamate binds to the extracellular portion of the receptor and provokes a response-excitotoxicity. Overstimulation of glutamate receptors causes neurodegeneration and neuronal damage through a process called excitotoxicity. Excessive glutamate, or excitotoxins acting on the same glutamate receptors, overactivate glutamate receptors, causing high levels of calcium ions (Ca²⁺) to influx into the postsynaptic cell. High Ca²⁺ concentrations activate a cascade of cell degradation processes involving proteases, lipases, nitric oxide synthase, and a number of enzymes that damage cell structures often to the point of cell death (Manev H, Favaron M, Guidotti A, Costa E (July 1989). “Delayed increase of Ca2+ influx elicited by glutamate: role in neuronal death”. Mol. Pharmacol. 36 (1): 106-12). Glutamate excitotoxicity triggered by overstimulation of glutamate receptors by light in the photoreceptors and RPE also contributes to intracellular oxidative stress. Proximal glial cells, in this case Muller cells use a cystine/glutamate antiporter to transport cystine into the cell and glutamate out. Excessive extracellular glutamate concentrations inhibits synthesis of glutathione (GSH), an antioxidant due to lack of enough cystine. Lack of GSH leads to more reactive oxygen species (ROSs) that damage and kill the glial cell Muller cells and photoreceptors, which then cannot reuptake and process extracellular glutamate (Markowitz A J, White M G, Kolson D L, Jordan-Sciutto K L (July 2007). “Cellular interplay between neurons and glia: toward a comprehensive mechanism for excitotoxic neuronal loss in neurodegeneration”. Cellscience 4 (1): 111-146). In addition, increased C^{a2+} concentrations activate nitric oxide synthase (NOS) and the over-synthesis of nitric oxide (NO). High NO concentration damages mitochondria, leading to more energy depletion, and adds oxidative stress to the photoreceptor neuron as NO is a ROS. In addition, cell death via lysis or apoptosis releases cytoplasmic glutamate outside of the ruptured cell. These two forms of glutamate release cause a continual domino effect of excitotoxic cell death and further increased extracellular glutamate concentrations.

Glutamate receptors' significance in excitotoxicity links it to many neurodegenerative diseases so also in AMD. Glutamate is almost exclusively located inside the cells. This is essential because glutamate receptors can only be activated by glutamate binding to them from the outside. Hence, glutamate is relatively inactive as long as it is intracellular. Hence, AMD is related to excessive glutamate stimulation of RPE, Muller cells and photoreceptors. Ketamine is one of most important NMDA blocker, thus prevent the excitotoxicity. The micro doses of ketamine we use in the ophthalmic drops have no hallucinogenic or other ill effect at all. It is one of the ideal ophthalmic therapeutic agents for treatment of various retinal diseases including AMD. Pharmacologically, ketamine is classified as an NMDA receptor antagonist. The present inventor has used this in thousands of case as dissociative anesthesia, neuropathic pain, depression, and experiment show that it inhibits the rabies virus multiplication. The invention described herein incorporates ketamine in the ophthalmic drops delivered to the conjunctional sac. It is important to note that ketamine has mil local anesthetic effect and thus prevents the stinging-burning experienced after conjunctional sac instillation of therapeutic agents.

Prophylaxis against AMD: Our observation suggests that the cholesterol in the fine capillaries supplying the macula lutea gets trapped as the mass of cholesterol micro particles. With aging, they gradually coalesce, and grow to form big cholesterol globules with incorporation of other particulate matter from the blood and presented as yellow drusen. With passage of time, other components of the blood is incorporated into this cholesterol mass, cutting of the blood and oxygen supply to the region resulting in RPE degeneration, angiogenesis (in wet AMD), photoreceptors apoptosis and other changes. That is why, as part of prophylaxis, and to curtail the further advancement of the AMD, we put all patients above the age of 55-60, on statins, green leafy vegetable diet, cutting down the intake of saturated fats and aerobic exercise and insulin ophthalmic drops. If there is hint of angiogenesis, the patients receive the prescription for low dose monoclonal antibodies ophthalmic drops as described. To reduce the production of ROS, patient advised to wear a cold pack on both eyes as they go to sleep.

Preliminary Preparation and Precautions Taken Treating the Age Related Macular Degeneration Patients Using Insulin and Adjuvant Therapeutic Agents Described in this Invention

Examination of the Patients Eyes Before Treatment

Before using described inventive methods and examples, a thorough examination of the AMD affected patient's eye is in order. The examination of the eye may include:
a) Acuity testing
b) Biomicroscopy
c) Intraocular pressure (IOP)
d) Ophthalmoscopy
e) Color vision test
f) Tear osmolality
g) Schimer's test
h) Tear film breakup time (tBUT)
i) Test for Superficial punctate keratitis (SPK)
j) Fluorescein and Rose Bengal staining (RBS) of BV of the retina, as well as cornea, conjunctiva, and eyelids
k) Slit-lamp examination of the conjunctiva, cornea, anterior chamber, iris, and lens
l) The Ocular Surface Disease Index (OSDI)
m) Microscopic examination of the tear filament
n) Maturation index (a Papanicolaous stained sample of conjunctival epithelium)
o) Important test for AMD and retinitis pigmentosa is electroretinogram (ERG) to measure the function of the photoreceptors.
p) In addition, a complete physical examination with blood test for thyroid, parathyroid, growth hormone, insulin, FSH, LH, cortisol, estradiol, and testosterone levels, electrolytes, blood cell count, cholesterol level, ESR, and a urine sample for pregnancy test when this is deemed necessary when the patient is of childbearing age. Select the test according to the eye diseases and their diagnosis.

Only selected test form the above list performed depending upon the oculopathy. To apply our inventive ophthalmic insulin drops as therapeutic agents, the patient or the caregiver has to wash their hands with a mild antiseptic soap. The person or patient applying the drops must be careful not to touch the dropper tip to the eyelids (and the foreign objects) to avoid contamination if there is an eyelid infection. Tilt the head back, or lay down with head extended on a neck pillow, gaze upward and backwards, and pull down the lower eyelid to expose the conjunctival fornix. Place the dropper directly over the eye away from the cornea and instill the prescribed number of drops. Look downward and gently close your eyes for 1 to 2 minutes. The patient should not rub the eye. Do not rinse the dropper unless the patient or person knows the sterilization technique with hot water. If other therapeutic, pharmaceutical, biochemical and biological agents or compounds are to be selected to treat the condition with our invention; the patient should wait at least 3-5 minutes before using other selected anti-age related macular degeneration therapeutic agents or the other variety of ophthalmic medicaments. It is important to instill medications regularly as prescribed to control age related macular degeneration. Consult your doctor and/or pharmacist if the systemic medications that you are taking are safe to use with the eye drops described and prescribed. When there is no contraindication for the insulin eye drops, you can treat patients, except, the patients with hypoglycemia syndromes and in some cases external ocular tumors.

To minimize the absorption into the bloodstream and to maximize, the amount of drug absorbed by the eye, close your eye for one to five minutes after administering the insulin drops. Then, press your index finger gently against the inferior nasal corner of your eyelid to close the tear duct, which drains into the nose (FIG. 10). This will prevent any adverse systemic effects due to nasal vascular uptake into the systemic circulation from the nasolacrimal duct drainage of the therapeutic agents from the conjunctival sac.

Eye drops may cause a mild uncomfortable burning or light stinging sensation, which this reaction should last for only a few seconds to minutes. The anti-age related macular degeneration drops take effect after 5-10 minutes after application depending upon the therapeutic agents used with the eye drops. We recommend that it is best to use insulin eye drops before bedtime and rising in the morning. This process can be repeated every 6, 12 or 24 hours for 3-7 days a week till the desirable results are obtained. Age related macular degeneration patients can use insulin eye drops all their lives or intermittently, depending on the results and the need. The therapeutic agents are instilled using a sterile dropper (or bottle with medication equipped with a dropper nipple) into the conjunctival sac.

Preparation of Insulin Eye Drops for Use in Age Related Macular Degeneration

Take 100 international units (IU) of rapid or intermediate or long acting insulin (or)) and dilute in 5 ml of sterile saline or distilled water which contains 0.01% povidone iodine with or without other carriers and facilitators as described above. The pH adjusted to prevent the sting when the insulin is dropped into the conjunctival sac using NaHCO₃. The preparation can contain nanograms (micrograms) of local anesthetics to prevent the stinging when the eye drops applied to the eye. In this preparation, each ml contains 20 units of insulin. That means each drop contains one unit of insulin.

In pharmacies, a drop was another name for a minim, which a drop would be 0.0616 milliliters. The drop standardized in the metric system to equal exactly 0.05 milliliters. The 20 drops equal one ml (1 cc) which each drop contains 0.10 IU of insulin. The concentration of the insulin content can be increased to 0.20, 0.30, 0.40, and 0.50 IU or even up to 1 or 2 or 3 unit of insulin per drop. The insulin content of the ophthalmic drops increased per drop in the dilutant preparation. The insulin content decreased by reducing the insulin units used for the preparation of the ophthalmic drops. Instill one to two drops to each eye lower lid fornix and/or everted upper eyelid (conjunctival sac) as a single agent. The applicant must apply pressure on the nasolacrimal duct as shown in the FIG. 10 to prevent drainage into the nasal cavity.

If other combinations of the anti-age related macular degeneration, therapeutic agents are used: first use insulin drops, wait for 3-5 minutes, and apply the other therapeutic, pharmaceutical, biochemical, and biological agents or compounds. After this procedure, instill one more insulin drop further enhance the uptake of the other selected therapeutic agents to augment-amplify their effects at the cellular level.

Principles of Compounding of Ophthalmic Insulin Drops to Enhance it Absorption, and Delivery to the Site of Pathology

Insulin compounded as a liquid ophthalmic isotonic solution containing therapeutic agents with one or more buffering agents, said buffering agents producing a pH in said composition similar to mammalian eye fluids.

The above pharmaceutical eye drop preparation of our invention may contain antibacterial components which these components are non-injurious to the eye when used. Examples are thimerosal, benzalkonium chloride, methyl and propyl paraben, benzyldodecinium bromide, benzyl alcohol, or phenyl ethanol. There is an autism controversy which we will avoid using thimerosal.

The therapeutic pharmaceutical preparation may contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The therapeutic, pharmaceutical, biochemical, and biological agents or compounds used in our invention may also contain a non-noxious pharmaceutical carrier, or with a non-toxic pharmaceutical inorganic substance. Typical of pharmaceutically acceptable carriers are, for example: water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, olyvinylpyrrolidone, isopropyl myristate and other traditionally acceptable carriers.

The therapeutic preparation may contain non-toxic emulsifying, preserving, wetting agents, and bodying agents. For example: polyethylene glycols 200, 300, 400 and 600, carbowaxes 1,000, 1,500, 4,000, 6,000 and 10,000, antibacterial components as quaternary ammonium compounds, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Furthermore, appropriate ophthalmic vehicles can be used as carrier media for the current purpose. This includes conventional phosphate buffer vehicle systems which are isotonic boric acid vehicles, isotonic sodium chloride vehicles, isotonic sodium borate vehicles and the like.

The objects accomplished by treating the eye with an aqueous composition containing an effective amount of a nonionic surfactant and insulin. The applicant has found that an effective amount of surfactant may comprise anywhere from 0.5 percent by weight and by volume to about 10 percent by weight and volume (hereinafter %), preferably about 1-5%, of active surfactant (not combined with oil) in the composition combined with insulin. However, the use of any oil in the composition will reduce the effectiveness of the surfactant.

The reason is that a substantial percentage of the surfactant tends to serve as a vehicle for dissolving or forming an emulsion of the oil with the aqueous layer to “wash” or hydrate the corneal surface. Thus, any oil is used in the composition, then, additional surfactant will be required to provide the effective amount of 0.5-10% preferably 1-5% of available active nonionic surfactant.

The anti-age related macular degeneration therapeutic agents' preparation may contain surfactants such as polysorbate surfactants, polyoxyethylene surfactants (BASF Cremaphor), phosphonates, saponins, and polyethoxylated castor oils. The preference is the polyethoxylated castor oils, which are commercially available.

The pharmaceutical preparation may contain wetting agents which the agents are already in use in ophthalmic solutions such as carboxy methyl cellulose, hydroxypropyl methylcellulose, glycerin, mannitol, polyvinyl alcohol or hydroxyethylcellulose. The diluting agent may be water, distilled water, sterile water, or artificial tears. The wetting agent is present in an amount of about 0.001% to about 10%.

The ophthalmic formulation of this invention may include acids and bases to adjust the pH, tonicity imparting agents such as sorbitol, glycerin and dextrose, other viscosity imparting agents such as sodium carboxymethylcellulose, polyvinylpyrrdidone, polyvinyl alcohol, and other gums. The suitable absorption enhancers are surfactants, bile acids. The stabilizing agents are antioxidants, like bisulfites and ascorbate. The metal chelating agents like sodium EDTA and drug solubility enhancers, which are the polyethylene glycols. These additional ingredients help give commercial solutions stability to the ophthalmic drops compounded.

Ophthalmic medications compositions will be compatible with the eye and/or contact lenses. The eye drop preparation should be isotonic with blood. The ophthalmic compositions, which are intended for direct application to the eye, will be formulated to have a pH and tonicity which these are compatible with the eye. This will normally require a buffer to maintain the pH of the composition at or near physiologic pH (i.e., pH 7.4) which the buffer may require a tonicity agent to bring the osmolality of the composition to a level or near 210-320 millimoles per kilogram.

The eye drop composition of the invention includes buffering agents to adjust the acidity or the alkalinity of the final preparation to prevent eye irritation. The composition is an isotonic solution in that it has the similar pH to fluids indicating that the pH of the composition is 6.1, 6.3, or 7.4. The buffering agents may include all of zinc sulfate, boric acid, and potassium necessary to be effective in achieving the pH of the composition of from 6.10 to 6.30, and to 8.00 typically. The total amount of buffering agents present in the composition ranges from 1% to 10% by weight of the composition.

The eye drop composition includes a lubricant such as cellulose derivatives (carboxymethyl cellulose). The composition may contain known preservatives conventionally used in eye drops such as benzalkonium chloride and other quaternary ammonium preservative agents, phenyl mercuric salts, sorbic acid, chlorobutanol, disodium edentate (EDTA), thimerosal, methyl and propyl paraben, benzyl alcohol, and phenyl ethanol. Purified benzyl alcohol may be in the concentration preferably from 0.1% to 5% by weight.

The eye treatment composition of the invention is a solution having a vehicle of water or mixtures of water and water-miscible solvents. For example, lower alkanols or arylalkanols, the phosphate buffers vehicle systems and isotonic vehicles where the vehicles are boric acid, sodium chloride, sodium citrate, sodium acetate and the like, vegetable oils, polyalkylene glycols, and petroleum based jelly, as well as aqueous solutions containing ethyl cellulose, carboxymethyl cellulose, and derivatives thereof. The hydroxypropylmethyl cellulose, hydroxyethyl cellulose, carbopol, polyvinyl alcohol, polyvinyl pyrrolidone, isopropyl myristate, and other conventionally employed non-toxic, pharmaceutically acceptable organic and inorganic carriers.

The composition is applied to the eye should be sterile in the form of an isotonic solution. The constitution may contain non-toxic supplementary substances such as emulsifying agents, wetting agents, bodying agents, and the like. For example, polyethylene glycols, carbowaxes, and polysorbate 80 and other conventional ingredients can be employed such as sorbitan monolaurate, triethanolamine, oleate, polyoxyethylene sorbitan 35 monopalmitylate, dioctyl sodium sulfosuccinate, monothioglycerol, thiosorbitol, ethylenediamine tetraacetic acid, and like.

The Following are the Examples of Using Our Invention of Insulin biological factors and in combination with known therapeutic, Pharmaceutical, Biological, Biochemical, Compounds and Nuteicuetical to Treat Age Related Macular Degeneration and Other Associated Retinal Diseases.

### Example 1

Select the patient; establish the type of Age related macular degeneration and its etiology, if possible, which the person is suffering from. The complete and thorough examination of the eye as described above is imperative. Record the preliminary examination results on the patient chart. The patient examined for any corneal, conjunctival, and retinal BV afflictions by using marker dyes and other ophthalmological examinations.
I. Position the patient in a supine posture or sitting with the head hyper extended with a support.
II. Prepare 0.05% povidone iodine in normal saline. Instill one or two drops to the conjunctional sac, wait 5 minutes for it to act on conjunctival lining and oxidize reduced glutathione to prevent it breaking the disulfide bonds of insulin.
III. Using a dropper or dropper bottle containing the insulin formulations. Insulin is prepared in 5 ml normal saline insulin dropper or plastic squeeze instiller. Instill two or three drops of insulin preparation in each eye lower lid fornix and/or everted upper eyelid (FIG. 1).
IV. Apply slight pressure at the nasal angle of eye on the nasolacrimal canaliculi-sac-duct system to prevent leaking of the therapeutic agents to the nose to avoid systemic absorption (FIG. 10). The adverse effects of insulin aborption can be prevented or minimized using the method shown in the FIG. 10.
V. The patient remain stationary for 3 to 5 minutes in supine position with head extended. The patient can resume the desired posture after the patient has been stationary for 5 minutes.
VI. The above instructions given to all the patients and caregivers. The patient or the caregiver trained to apply the ophthalmic drops using sterile methods. The insulin ophthalmic therapeutic drops used before going to bed and after getting up from bed in the morning, after taking a shower as well as before taking a nap in the afternoon if possible and during daytime apply the drops every 8 hours.

Case reports: This is a 68-year-old male patient came for the treatment of lung cancer. He had vision problems and diagnosed as early case of dry AMD. The patient provided with the insulin ophthalmic drops used as above described. After two weeks of use, patients reported improve vision and after 3 months of use, he had good vision and could drive. Patient succumbed to the heart disease 9 months later.

This is a 62-year-old female patient diagnosed with dry AMD. She also suffered from dry eye syndrome. She was prescribed with cyclocsporin drops (Restasis™, Allergan, Inc., and Irvine, Calif.) for dry eyes condition. We prepared and provided her insulin ophthalmic drops as described in example 1. We advised her to use cyclosporin drops first, wait for 3-5 minutes, and then instill insulin ophthalmic drops as described above. After 4 weeks of use, her symptoms of AMD decreased, vision improved, and at the same time, the dry eyes symptoms reduced. She reported that the daily use of restasis for dry eye symptoms reduced and uses once or twice a day.

### Example 2

This is a 70-year-old patient diagnosed with wet AMD in right eye with CNV, associated with slight edema. The left eye had early symptoms of dry AMD, with still had good vision. It has had Drusen deposits in the macula, but no angiogenesis. The patient refused to undergo once every six-week intravitreal injection of anti-angiogenesis monoclonal antibody, Bevacizumab (trade name AVASTIN™, Genentech/Roche). AVASTIN (bevacizumab) is a recombinant humanized monoclonal IgG1 antibody that binds to and inhibits the biologic activity of human vascular endothelial growth factor (VEGF) in vitro and in vivo. It blocks angiogenesis, the growth of new blood vessels. This therapeutic agent used in doses of 8.3 to 10 mg in 0.3 ml solution injected in to the vitreous. It is not FDA approved for treating wet AMD, but many ophthalmologists use it off label. One of the advantages of these monoclonal antibodies is that it many times less expensive compared to another FDA approved monoclonal antibodies Ranibizumab (LUCENTIS™) for the treatment of wet AMD, which is a smaller molecule and said to permeate easily compared to Bevacizumab.

The patients were afraid of sticking the needle in the eye every six week. We have used this monoclonal antibody in treatment of advanced cancers in very large doses, but not to treat AMD. Bevacizumab inhibits vascular endothelial growth factor A (VEGF-A). VEGF-A is a chemical signal that stimulates angiogenesis in a variety of diseases, especially in cancer and in AMD. Bevacizumab was the first clinically available angiogenesis inhibitor in the United States. Bevacizumab used to treat various cancers, such as colorectal, lung, breast, kidney, and glioblastomas.

Bevacizumab is a clear to slightly opalescent, colorless to pale brown, sterile, pH 6.2 solutions for intravenous infusion. It supplied in 100 mg and 400 mg preservative-free, single-use vials to deliver 4 mL or 16 mL of AVASTIN (25 mg/mL). The 100 mg product is formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and Water for Injection, USP. The 400 mg product is formulated in 960 mg α,α-trehalose dihydrate, 92.8 mg sodium phosphate (monobasic, monohydrate), 19.2 mg sodium phosphate (dibasic, anhydrous), 6.4 mg polysorbate 20, and Water for Injection, USP.
I. First use the insulin drops and wait for 5 minutes as described in example 1.
II. Take 100 mg in 4 ml vial of Bevacizumab, which means each ml contains 25 mg. Draw 0.3 ml, which containing 8.3 mg of the monoclonal antibodies in an insulin syringe. After drawing the solution, discard the needle. Now, one has the monoclonal antibodies in the syringe and it acts as a dropper. Then instill 0.3 ml of Bevacizumab (8.3 mg) into the conjunctival sac in increments of drops to prevent spill over with patient in supine position with head extended. Take 60-90 minutes to complete the instillation. It is similar time taken for to IV infusion for cancer patients.
III. Following this instillation completed, wait for 30 minutes in supine position with head extended on supporting pillow.
IV. Following Bevacizumab delivery, instill insulin preparation in to the conjunctival sac as explained in example 1. Wait for 15 minutes for it to be completely absorbed.
V. The patient stays in a quiet room resting for 30 more minutes and then sent home with a supporting driver or Taxi.
VI. At home, the patient instructed to instill insulin drops every 6-8 hourly. The patient advised to rest for 8 hours if possible in supine position.

Complications: There were no systemic complications as described for treatment of cancers using Bevacizumab. It is because, the dose used as ophthalmic drops is one to two hundredths that used in the treatment of cancers.

Precautions: Do not use Bevacizumab or any other monoclonal antibodies drops if there is recent surgery of the eye or corneal and conjunctional lining scratches. Wait until there is complete healing, usually up to 28 days. Avoid using any contact lens (which is rare in the aged) when undergoing this treatment. Do not dilute the Bevacizumab in dextrose. Use normal saline to dilute it.

The therapy repeated every two weeks until improvement seen, then every four to six weeks. After 4 therapies, the patient showed improvement in vision. The vascular plexus around the macula lutea began to shrink with reduced swelling. These patients prescribe statin drugs in addition.

### Example 3

I. Follow the instruction as described in the above EXAMPLE 1.
II. Instead of using Bevacizumab, use Ranibizumab monoclonal antibodies ophthalmic drops in similar way as described in example 2.

### Example 4

Antibodies are proteins generated by the immune system's white blood cells. The antibodies circulate in the blood and attach to foreign proteins called antigens in order to destroy or to neutralize them. By this mode, the antibodies help rid the systemic infection or eliminate foreign proteins (non-self) harmful to the body cells. Monoclonal antibodies are laboratory created or fashioned substances that the antibodies can locate and bind to them and make them ineffective. The antibodies bind to specific molecules such as tumor necrosis factor (TNF) which the TNF is a protein involved in causing the inflammation and the damage of autoimmune diseases.
I. The etiology of AMD and wet AMD blamed on possibly autoimmune type inflammation resulting in activation of VEGF to produce new unwanted BV in the macula lutea. Besides blocking the VEGF as described in example 2, it is also important to block inflammatory stimulus. There are many monoclonal antibodies (mAB)) such as: REMICADE™, etanercept, EMBREL™, and HUMIRA™. The anti TNF agents are on the market to treat a dozen or so autoimmune diseases such as rheumatoid arthritis, psoriasis's, scleroderma and such diseases'. Etanercept is such a mAB used to treat autoimmune diseases by interfering with the tumor necrosis factor (TNF, a part of the immune system) by acting as a TNF inhibitor. This therapeutic potential is based on the fact that TNF-alpha is the “master regulator” of the inflammatory response in many organ systems and is a cytokine produced by lymphocytes and macrophages.
II. Multiple monoclonal antibodies are currently under investigation for the treatment of age related macular degeneration (Meijer J M, Pijpe J, Bootsma H, Vissink A, Kallenberg C G (June 2007). “The future of biologic agents in the treatment of “Sjögren's syndrome”. Clin Rev Allergy Immunol 32 (3): 292-7). All TNF inhibitors are immune-suppressants. We formulate Etanercept (Embrel) to treat inflammation of the contributing to AMD. ETANERCEPT is a dimeric fusion fusion protein produced through expression of recombinant DNA. That is, it is a product of a DNA “construct” engineered to link the human gene for soluble TNF receptor 2 to the gene for the Fc component of human immunoglobulin G1 (IgG1). Expression of the construct produces a continuous protein “fusing” TNF receptor 2 to IgG1. Production of Etanercept is accomplished by the large-scale culturing of cells that have been “cloned” to express this recombinant DNA construct. We selected ETANERCEPT, because it is has been available long time and used extensively.
   I. ETANERCEPT supplied in a 25 mg multiple-use vial as a sterile, white, preservative-free, lyophilized powder. Reconstitution with 1 mL of the supplied Sterile Bacteriostatic Water for Injection, USP (containing 0.9% benzyl alcohol) yields a multiple-use, clear, and colorless solution with a pH of 7.4±0.3.
   II. Preparation of ophthalmic drops: take 25 mg solution and make 5 ml in the bacteriostatic water. Each ml contains 5 mg of the Etanercept. Take one ml of this stock solution and mix with 5 ml of distilled water, each ml contain 1000 micrograms of the monoclonal antibody. Each drop will contain 50 mcg of the active ingredient.
   III. Follow the instruction as described in the above EXAMPLE 2.
   IV. Use the ETANERCEPT monoclonal antibody using no more than 1000 μg per ml of ophthalmic solution, which results in 50 μg per drop instilled. Instill 2-3 drops to each eye. Wait for 15 minutes in supine head extended looking to the ceiling to get absorbed. Prevent the overflow of the drops.
   V. When the conjunctival sac is free of ETANERCEPT, then instill insulin.
   VI. Repeat the process 2-3 times a day.
   VII. Instruct the patient to store the Etanercept in refrigerator, not freezer.
   VIII. Instruct the patient to use at bedtime before going to sleep every night.

We must take into account any contraindications such as tuberculosis or tumors while using these biological therapeutic agents with this insulin invention. The dose we use is too small and has no systemic spread to cause toxicity. Only contraindication of ETANERCEPT monoclonal antibodies is any recent surgery or injury to the eye and history of eye tumors.

Case report: This is a 60-year-old male patient. He has early symptoms of AMD in both eyes. His history revealed that he has eaten two eggs with bread coated with real butter for 4 decades. He developed vision problems. Eye examination showed the Drusen deposits around macula lutea but no CNV. They were not coalesced to from a thick ring around the macula lutea as seen advanced cases of AMD. Diagnosis of dry AMD made. He has difficulty in nighttime driving also. He could not read the road signs easily. He was treated with the above regimen. He was put on low fat high lutein green leafy vegetable diet with vitamin supplements. We prescribed Lipitor 80 mg taken daily before going to bed. His liver enzymes were within normal levels. His cholesterol went down after one month of therapy, his vision improved considerably, and drusen deposits became smaller. His difficulty of nighttime driving improved and could read the road signs better than before.

### Example 5

According to Catharine Gale et al, U.S. Patent Application Publication Number: 2003/0065020), in a cross-sectional survey of men and women, that use of statins is associated with an 11-fold reduction in risk of macular degeneration. This study reveals to us that the hypercholesterimia is directly linked to the production of drusen (as described above), which disrupts the nutritional supply to macula lutea and hypoxia resulting in dry AMD and angiogenesis leading to wet AMD. Statins are inhibitors of 3-hydroxy-3-methylglutaryl coenzyme A, i.e. HMG-CoA reductase inhibitors. They reduce LDL and increase HDL. Consequently, less cholesterol end up around the macula lutea surrounding BV. Accordingly, we provide that age-related macular degeneration (AMD) is effectively treated by administration of HMG-CoA reductase inhibitors such as statins followed by insulin ophthalmic drops.
I. Follow the instruction as described in the above EXAMPLE 1.
II. All our patient with high level of blood cholesterol and vision problems were put on Lipitor 80 mg at bed time, depending upon the blood cholesterol levels after liver enzyme analysis.
III. Then, they were put on strict regimen of low cholesterol, no saturated fat, once a month red meat, weekly three times fish diet and dairy product free with green leafy vegetables in the diet. Prescribe and provided insulin drops to at the bedtime and one time in the daytime.
IV. Advised a regimen of aerobic exercise to reduce cholesterol, increase oxygen supply to the ocular structures to prevent hypoxia of macula which can initiate angiogenesis to cause wet AMD.
V. All most all the patients had reduction in blood cholesterol, vision improved, and there were no more addition of drusen deposits, and the large drusen begin to shrink.

### Example 6

Pegaptanib (MACUGEN™): Pegaptanib is a pegylated anti-VEGF aptamer. Aptamer are oligonucleic acid or peptide molecules that bind to a specific target molecule. Aptamers created by selecting them from a large random sequence pool. Natural aptamers also exist in riboswitches, a single strand of nucleic acid that binds with specificity to VEGF 165. This latter protein plays a critical role in angiogenesis (the formation of new blood vessels) and increased permeability (leakage from blood vessels-causing macular edema), two of the primary pathological processes responsible for the vision loss associated with neovascular wet AMD. The FDA approved Pegaptanib (MACUGEN™) to treat wet macular degeneration in December 2004. MACUGEN is injected into the eye every six weeks, in 0.3 mg doses at a time six weekly according to the American Macular Degeneration Foundation website. MACUGEN slows down visual loss from wet macular degeneration. Pegaptanib decreases the level of a protein that affects the cells of the eye. This protein can cause swelling and blood vessel changes that lead to macular degeneration and blindness.

Case report: This is a 72 year old patients diagnosed with wet AMD with vision changes. There was swelling of the macula lutea, with not much predominant BV plexus formation. The patient treated with MACUGEN as described in Example 2. The dose 0.3 mg diluted in 1 ml of saline used at each sitting. The procedure repeated every two week once. He put on hydrochlorothiazide diuretics to remove excess fluid in tissue spaces to reduce the edema of macula lutea. The patient's vision improved and the swelling of the macula lutea also decreased considerably.

### Example 7

There are other drugs used to treat cancer, such as thalidomide (THALOMID®) and lenalidomide (REVLIMID®), known to act as inhibitors of new blood vessel growth. We treated cancers with thalomid to prevent metastasis and growth by inhibiting angiogenesis for more than a decade with excellent positive outcome. These drugs not used for treatment of wet AMD so far. Lenalidomide marketed as REVLIMID® by Celgene, is a derivative of thalidomide, induces tumor cell apoptosis directly, anti-angiogenic, and has immune-modulator activity. Lenalidomide has a broad range of activities and used successfully to treat both inflammatory disorders and cancers in the past 10 years. We have used this therapeutic agents only once in a case of wet AMD with insulin. The ophthalmic preparation with insulin can be of immense value in the treatment of wet AMD associated with ocular tumors. We plan to use these therapeutic agents as ophthalmic drops with Insulin in our invention to treat wet AMD and other diseases of the eye associated with angiogenesis such as diabetic retinopathy, wet AMD and vascular tumors of the eye.

### Example 8

Eliminate Glutamate toxicity in the prevention and treatment of AMD using insulin and ketamine: Glutamine (Gln), glutamate (Glu) and γ-amino butyric acid (GABA) are essential amino acids for brain metabolism and function. Glutamate is synthesized from glutamine in glutamatergic neurons via the action of the enzyme glutaminase and, following synaptic release, is removed into both nerve terminals and glial cells by selective energy-dependent transporters. Glial cells subsequently reconvert glutamate into glutamine, via the enzyme glutamine synthetase, and glutamine is finally transferred to glutamatergic neurons, completing the so-called glutamate-glutamine cycle. Glutamate homeostasis is critical to the normal functioning of the nervous system, retina, and in this regard, glial glutamate uptake is believed to be of principal importance. Glutamate is not only a neurotransmitter but also an excitotoxic agent that, in high concentrations, has the potential to cause cell death.

According to a model known as the excitotoxicity theory, lower energy levels in the nerve cells and photoreceptors of people with AMD and retinitis pigmentosa, cause them to be overly sensitive to glutamate. Consequently, even normal levels of glutamate can over activate the glutamate receptors on the nerve cells. When these receptors (also known as NMDA receptors) activated, calcium ions enter the nerve cells. Excessive activation causes a buildup of these calcium ions, which then leads to the death of the nerve cell in the brain and retina. Drugs like ketamine and Memantine are also a non-competitive antagonist. “Non-competitive” means that they bind to a site on the NMDA receptor that is different from glutamate's binding site. By binding to one portion of the NMDA receptor, these therapeutic agents' changes, the overall shape of the receptor, and making it more difficult for glutamate to bind to the other portion of the receptor to initiate excitotoxicity. As a result, it is maintained at low levels in the “extracellular fluid of the brain by efficient, but energetically expensive uptake into glial cells astrocytic-derived and in the eyes Muller cells.

Glutamine is the precursor of the two most important neurotransmitters: glutamate, an excitatory neurotransmitter, and GABA, an inhibitory neurotransmitter. Glutamine is a derivative of glutamic acid. Its chemical name is glutamic acid 5-amide. In addition to their roles in neurotransmission, these neurotransmitters act as alternative metabolic substrates that enable metabolic coupling between glial cells such as astrocytes, Müller cells, and neurons.

Glutamate is a powerful excitatory neurotransmitter released by nerve cells in the brain and retina. It is responsible for sending signals between nerve cells, and under normal conditions, it plays an important role in learning and memory. There are two general ways, however, that glutamate can actually be damaging to nerve cells and the brain as a whole including retina—an extension of the brain. First, there can be too much glutamate around; abnormally high concentrations of glutamate can lead to over excitation of the receiving nerve cell. Second, the receptors for glutamate on the receiving nerve cell can be oversensitive, such that less glutamate molecules are necessary to excite that cell. These mechanisms may play an important role in damaging already defective photoreceptors and RPE in AMD and retinitis pigmentosa. Further, the reactive oxygen species (ROS) produced due to light perceptions and hypoxia (due to Drusen deposits) in the aged retina (AMD), results in complete deregulation of RPE, Müller cells and photoreceptors metabolism leading to ARM. Research shows that glutamate receptors are present in CNS glial cells as well as neurons, so also retina including RPE and Müller cells, which can act as excitotoxic to the sensitive photoreceptors. The glutamate binds to the extracellular portion of the receptor and provokes a response-excitotoxicity. Overstimulation of glutamate receptors causes neurodegeneration and neuronal damage through a process called excitotoxicity. Excessive glutamate, or excitotoxins acting on the glutamate receptors, and over activate glutamate receptors, causing high levels of calcium ions (Ca²⁺) to influx into the postsynaptic cell and photoreceptors. High Ca²⁺ concentrations, activate a cascade of cell degradation processes involving proteases, lipases, nitric oxide synthase, and a number of enzymes that damage cell structures often to the point of cell death (Manev H, Favaron M, Guidotti A, Costa E (July 1989). “Delayed increase of Ca²⁺ influx elicited by glutamate: role in neuronal death”. Mol. Pharmacol. 36 (1): 106-12).

Glutamate excitotoxicity triggered by overstimulation of glutamate receptors also contributes to intracellular oxidative stress. Glial cells such as Müller cells use a cystine/glutamate antiporter to transport cystine into the cell and glutamate out. Excessive extracellular glutamate concentrations inhibits synthesized glutathione (GSH), an antioxidant due to lack of enough cystine. Lack of GSH leads to more reactive oxygen species (ROSs) that damage and kill the glial and neuronal cell, which then cannot reuptake and process extracellular glutamate (Markowitz A J, White M G, Kolson D L, Jordan-Sciutto K L (July 2007). “Cellular interplay between neurons and glia: toward a comprehensive mechanism for excitotoxic neuronal loss in neurodegeneration”. Cell science 4 (1): 111-146). In addition, increased Ca²⁺ concentrations activate nitric oxide synthase (NOS) and the over-synthesis of nitric oxide (NO). High NO concentration damages mitochondria, leading to more energy depletion, and adds oxidative stress to the photoreceptors and neuron as NO is a ROS. In addition, cell death via lysis or apoptosis releases cytoplasmic glutamate outside of the ruptured cell. These two forms of glutamate release cause a continual domino effect of excitotoxic cell death and further increased extracellular glutamate concentrations. The ischemia of the eyes due to excess build up of drusen in macula lutea vessels and other etiologies, leads to an excessive activation of glutamate receptors, which lead to photoreceptors injury and apoptosis.

Glutamate is exclusively located inside the cells. The intracellular location of some 99.99% of brain and retinal glutamate is the reason why this system can work. This is essential because glutamate receptors can only be activated by glutamate binding to them from the outside. Hence, glutamate is relatively inactive as long as it is intracellular. The photons of light, hypoxic damage due to ASVD and drusen built up, genetic predisposition contribute to glutamine release from the retina, resulting in pathological changes and apoptosis in the photoreceptors, RPE, Muller cells, and choriocapillares leading to AMD.

Ketamine is a GABA receptors antagonist. It acts by blocking the N-methyl-D-aspartic acid (NMDA) receptor, which receives signals from glutamate. There are many examples of antagonists of the NMDA receptor such as Amantadine, dextromethorphan, ketamine, phencyclidine (PCP), riluzole, memantine, and kynurenic acid; the latter is the only known endogenous antagonist. They referred to as NMDA receptor antagonists. Ketamine is the dissociative anesthetic, excellent sedative, it is an anti arrhythmic, reduces the pain perception due to its local anesthetic like effects, maintains bronchial dilatation, does not decrease the BP, and causes tachypnoea, with the inhibition of rabies virus multiplication and blocks the NMDA receptors. We have used ketamine for dress changing in burn patients since 1969 and postpartum—after delivery to ally the anxiety under regional anesthesia, treatment of mental depression in terminal patients for 3 decades. Ketamine acts as a local anesthetic. According to the “gate theory of pain” of Melzack and Wall, gate theory, increased central efferent impulses can act on the gate (located in the spinal cord) and close the gate system (no feeling of pain) for all input from any site on the body (Melzack R, Wall P D: Pain mechanisms: a new theory. Science 150:971-979, 1965). It has been used for hiccup after surgery by IV administration (Shantha, T. R. Ketamine for the Treatment of Hiccups During and Following Anesthesia: A Preliminary Report in Anesthesia and Analgesia. Current Researches VOL. 52, No. 5, September-October, 1973. Dowdy E G, Kaya K, Gocho Y: Some pharmacologic similarities Of ketamine and local anesthetics. Abstracts of Scientific Papers, 1971 ASA Annual Meeting, p 165). There is evidence that neurotrophic viruses, including human immunodeficiency and rabies virus induces neuronal injury through N-methyl D-aspartate (NMDA) excitotoxicity mechanisms and that the (NMDA) receptor may be one of the rabies virus receptors (U.S. Patent Application Publication Number: 201110020279 AI. RABIES CURE, Totada R. Shantha). We have used these therapeutic agents extensively in cancer and Lyme diseases patients to ally pain, RSD, phantom limb syndrome, chronic neuropathic pain, and to reduce depression. Ketamin administered intranasal in these patients. We also have used it to treat early cases of both wet and dry AMD and retinitis pigmentosa.
a) Follow the instruction as described in the above EXAMPLE 1.
b) Apply insulin drops to the eye as explained in the example 1.
c) Take ketamin, Prepare 100 mcg per ml ketamin in saline. Then apply them into the conjunctional sac of the AMD afflicted eyes.
d) The rest of the instructions are as described in the example 1.

### Example 9

In this preparation, take 5 ml of normal saline. To each ml add:
a) short acting insulin 40 units
b) Chlorin e6, 20 mg
c) EDTA 30 mcg
d) Lidocaine hydrochloride 30 mcg
e) Prepare in a 5 or 10 ml sterile bottle with an eyedropper or plastic squeeze dropper. The dispenser is pre sterilized in boiling water or in a pressure sterilizer before mixing the above contents.
f) Mix them well in pharmaceutical shaker for 15 minutes under strict aseptic conditions and store in a clean cool refrigerator until used.
g) The composition can be dispensed as liquid drops, or as gel deposited under the eyelids instilled specially before going to sleep, then every 6-8 hourly during day time.
   Case report: This is a 65-year-old male diagnosed as early case of AMD. He went hunting in the wee hours of the morning, when the sun has not yet risen during deer hunting season. He had difficulty of vision at dusk. He used prescriptions glasses and had cataract surgery. He used the ophthalmic preparation as described above before going to bed and before going hunting and in the middle of the day. His night vision also improved according to subjective report and does not bump into objects as it used to happen in the home due to poor night vision. He could drive with less night vision problems. He uses the eye drops routinely at night before going to bed and before sunset. Chlorin e6 enhances the night vision perceptions through its incorporation into rods. Experiments have shown that the Chlorin e6, a chlorophyll derivative, gets incorporated to retinal photoreceptors and enhances the night vision in experimental subjects (Washington I, Jilin Zhou, Steffen Jockusch, Nicholas J. Turro, Koji Nakanishi and Janet R. Sparrow. Chlorophyll derivatives as visual pigments for super vision in the red. Photochem. Photobiol. Sci., 2007, 6, 775-779.).

Intravitreal stem cell injection and use of insulin ophthalmic drops for seeding and multiplication of stem cells in the retina: Attempts made with limited success by intravitreal injection of stem cells derived from fertilized human embryo, (not the umbilical cord stem cells). These stem cells are supposed to be seeded in the PRE and multiply to establish new RPE to replace the apoptic or dysfunctional RPE, may be even photoreceptors. We recommend these researchers to use insulin ophthalmic drops to support the multiplication of stem cells within the vitreous, and effectively seeded on the RPE. The theory is that the RPE is important to maintain the photoreceptors cells in the macula lutea and their death or dysfunction are the reason for the destruction of photoreceptors cells leading to the development of dry AMD. That means that the stem cells have to travel the complicated journey to reach selectively the RPE. This treatment is not applicable to wet AMD yet. This is because dry AMD does not involve the growth of abnormal new blood vessels. Research is underway to experiment and find out why the cells of the macula stop working and die.

Attempts made with limited success by intravitreal injection of stem cells derived from fertilized human embryo, (not the umbilical cord stem cells). These stem cells are to be seeded in the PRE and multiply to establish new RPE to replace the apoptic or dysfunctional RPE, may be even photoreceptors. We recommend these researchers to use insulin ophthalmic drops to support the multiplication of stem cells within the vitreous, and to be seeded on the RPE, photoreceptors and the rest of the retina. The theory is that the RPE is important to maintain the photoreceptors cells in the macula lutea and their death or dysfunction are the reason for the destruction of photoreceptors cells leading to the development of AMD. That means that the stem cells have to travel the complicated journey to reach selectively the RPE and photoreceptors. This treatment is not applicable to wet AMD yet. This is because dry AMD does not involve the growth of abnormal new blood vessels. We recommend the following regimen:
a) After injecting stem cells intravitrealy, wait for 12-24 hours for stem cells seeded in the retina and the give rest to the eyeball after this invasive procedure.
b) The insulin ophthalmic drops are prepared as described in example 1.
c) Instill insulin ophthalmic drops to the conjunctional sac as described above.
d) If there is no angiogenesis associated AMD, insulin drops combined with IGF-1, which is neurotrophic factor which helps in transformation of these stem cells to photoreceptors and RPE.
e) Apply the insulin with or without IGF-1 drops every 6-8 hourly and at bedtime.
f) Continue the ophthalmic application until the seeding and final differentiation of stem cells achieved after intravitreal injection of embryonic stem cells.
g) The use of ophthalmic insulin drops not only helps in the treatment of AMD, it also helps the embryonic stem cells to achieve desired results due to mitogenic effect of insulin.

Advantages of the Current Invention to Treat AMD

Advantage of the present invention is that the insulin in various varieties or forms, from synthetic or animal source is easily available.

The synthetic form is hypo-allergic without any untoward effect.

Any physician can prescribe these therapeutic agents.

Advantage of the present invention is that the insulin genetically synthesized for ophthalmic drops use.

An added benefit of the present invention is that it does away with the need for professional and laboratory assistance.

An added benefit of the present invention is that it provides a method where by insulin by itself have therapeutic effect in curtailing the AMD and enhancing the vision.

An added benefit of the present invention is that it provides therapeutic agents and insulin dispensed separately.

An added benefit of the present invention is that it provides therapeutic agents and insulin ophthalmic drops dispensed in a single dispenser.

An added benefit of the present invention is that both insulin and other therapeutic agents are easily available for treating AMD.

An added benefit of the present invention is that the preparations of these ophthalmic drops are not prohibitively expensive except one of the monoclonal antibody.

An added benefit of the present invention is that it avoids the intravitreal injection of monoclonal antibody, an invasive traumatic procedure and its associated ocular complications.

An added benefit of the present invention is that there are no short term or long term adverse effects on the eye.

There are no systemic effects using these therapeutic agents of this invention.

An added benefit of the present invention is that these ophthalmic drops used shortly after taking antioxidants and other vision enhancing agents such as Lutein and vitamins orally to augment and amplify their effect and alleviate or improve AMD.

Another advantage of this invention is that it instilled along with chlorin e6 to treat decreased night vision and night blindness, one of the distressing symptoms in cases age related macular degeneration and retinitis pigmentosa.

Another side benefit our invention is, it focuses on saving photoreceptors not affected by glutamate excitotoxicity and the fee radicals, in which they can be damaged by a spillover of free radicals, harmful metabolites, and biochemical products in the retina especially in cases of AMD and retinitis pigmentosa.

Another benefit of using this invention is that insulin which is widely available, inexpensive, and its therapeutic effects well established over a period of 90 years.

Yet another advantage of the present invention is that the use of insulin to enhance the uptake of the natural therapeutic agents when they reach the choroid and photoreceptors. Ophthalmic preparations are supplemented with oral intake of various retinal photoreceptors vision supporting lutein, and vitamin A rich nurticeuticals preparations such as blueberries, dihydroquercetin, beta-carotene (carrots), chlorella, lutein, Zeaxanthin, Omega 3 Oils (DHA+EPA), vitamins A, B1, B2, B6, B12, D₃ and metal zinc, the night vision will improve. Lutein and vitamin A with B complex supplements with insulin drops will enhance the vision, improve the night vision, and prevent the progression of the AMD.

Another advantage of our invention is that these eye drops compounded with other adjuvant therapeutic agents such as antioxidants, monoclonal antibodies, prostaglandins, antibiotics, chemotherapeutic agents, nerve growth factors, and hormonal preparations, which will cure or curtail the AMD, improve the night vision, reduce the night blindness, and treat oculopathies associated with or without these conditions.

Yet, another advantage of the present invention is that insulin augments and amplifies the therapeutic agents activity used to treat AMD.

A further plus of the present invention is that it provides therapeutic agents easily instilled into the eyes, stored, cleaned, and mass-produced economically to make it affordable for millions of aging population who can develop AMD.

A further plus of the present invention is, it provides therapeutic agents that can be easily used, with inulin, composition comprising at least one human growth factor selected from the group consisting of basic fibroblast growth factor (bFGF), glial-derived neurotrophic factor (CNTF), pigment epithelium-derived factor (PEDF), glial-derived neurotrophic factor (GDNF), and brain-derived neurotrophic factor (BDNF).

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made. The preferred embodiment of the present invention has been described. The invention should be understood that various changes, adaptations, and modifications may be made thereto. It should be understood, therefore, that the invention is not limited to details of the illustrated invention. This method can be used to diagnose and treat all the retinal diseases as well as prevent them. Although the instant invention has been described in relation to particular embodiments thereof, many other variations and modifications and other uses will become apparent to those skilled in the art.

What is claimed is:

1. A method of treating age related macular degeneration of an afflicted eye delivering adjuvant therapeutic agents through a conjunctival sac in vertebrates, to be delivered to the macula lutea, the site of the age related macular degeneration; comprising the steps of:
preparing an ophthalmic preparation of a 0.1% solution of povidone iodine in saline;
instilling said ophthalmic preparation into a conjunctional sac;
waiting 3-5 minutes; whereby said ophthalmic preparation is oxidized reducing glutathione to prevent an effect on insulin disulfide bonds;
placing a patient in a supine position with head slightly extended;
administering 2 to 3 insulin drops using an eyedropper or plastic squeeze dropper bottle containing an insulin therapeutic agent to said conjunctional sac; and
pressing on a naso-lacrimal canaliculi wherein drainage of said insulin drops is prevented.

2. The method of treating age related macular degeneration of an afflicted eye delivering adjuvant therapeutic agents through a conjunctival sac in vertebrates, to be delivered to the macula lutea, the site of the age related macular degeneration according to claim 1 wherein said vertebrate is a human.

3. The method of treating age related macular degeneration of an afflicted eye delivering adjuvant therapeutic agents through a conjunctival sac in vertebrates, to be delivered to the macula lutea, the site of the age related macular degeneration according to claim 1 wherein said vertebrate is a mammal.

4. The method of treating age related macular degeneration of an afflicted eye delivering adjuvant therapeutic agents through a conjunctival sac in vertebrates, to be delivered to the macula lutea, the site of the age related macular degeneration according to claim 1 wherein each milliliter of said insulin drop contains 20 IU of short acting insulin.

5. The method of treating age related macular degeneration of an afflicted eye delivering adjuvant therapeutic agents through a conjunctival sac in vertebrates, to be delivered to the macula lutea, the site of the age related macular degeneration according to claim 1 where in each milliliter of said ophthalmic preparation contains 15 IUs of insulin.

6. The method of treating age related macular degeneration of an afflicted eye delivering adjuvant therapeutic agents through a conjunctival sac in vertebrates, to be delivered to the macula lutea, the site of the age related macular degeneration according to claim 1 where in each milliliter of said ophthalmic preparation contains 10 IUs of insulin.

7. The method of treating age related macular degeneration of an afflicted eye delivering adjuvant therapeutic agents through a conjunctival sac in vertebrates, to be delivered to the macula lutea, the site of the age related macular degeneration according to claim 1 wherein each milliliter of said ophthalmic preparation contains between 0.05 to 1.0 percent povidone iodine dissolved in normal saline.

8. The method of treating age related macular degeneration of an afflicted eye delivering adjuvant therapeutic agents through a conjunctival sac in vertebrates, to be delivered to the macula lutea, the site of the age related macular degeneration according to claim 1 further comprising the step of instilling an antiangiogenic monoclonal antibody into said conjunctional sac.

9. The method of treating age related macular degeneration of an afflicted eye delivering adjuvant therapeutic agents through a conjunctival sac in vertebrates, to be delivered to the macula lutea, the site of the age related macular degeneration according to claim 8 wherein said antiangiogenic monoclonal antibody is bevacizumab.

10. The method of treating age related macular degeneration of an afflicted eye delivering adjuvant therapeutic agents through a conjunctival sac in vertebrates, to be delivered to the macula lutea, the site of the age related macular degeneration according to claim 8 wherein said antiangiogenic monoclonal antibody is etanercept.

11. The method of treating age related macular degeneration of an afflicted eye delivering adjuvant therapeutic agents through a conjunctival sac in vertebrates, to be delivered to the macula lutea, the site of the age related macular degeneration according to claim 8 wherein said antiangiogenic monoclonal antibody is ranibizumab.

12. The method of treating age related macular degeneration of an afflicted eye delivering adjuvant therapeutic agents through a conjunctival sac in vertebrates, to be delivered to the macula lutea, the site of the age related macular degeneration according to claim 1 further comprising the step of orally administering a medically effective dose of a statin.

13. The method of treating age related macular degeneration of an afflicted eye delivering adjuvant therapeutic agents through a conjunctival sac in vertebrates, to be delivered to the macula lutea, the site of the age related macular degeneration according to claim 1 wherein said adjuvant therapeutic agents are acetazolamide and brinzolamide.

14. The method of treating age related macular degeneration of an afflicted eye delivering adjuvant therapeutic agents through a conjunctival sac in vertebrates, to be delivered to the macula lutea, the site of the age related macular degeneration according to claim 1 wherein said adjuvant therapeutic agents is a corticosteroids.

15. The method of treating age related macular degeneration of an afflicted eye delivering adjuvant therapeutic agents through a conjunctival sac in vertebrates, to be delivered to the macula lutea, the site of the age related macular degeneration according to claim 1 wherein said adjuvant therapeutic agents is ketamine.

16. The method of treating age related macular degeneration of an afflicted eye delivering adjuvant therapeutic agents through a conjunctival sac in vertebrates, to be delivered to the macula lutea, the site of the age related macular degeneration according to claim 1 wherein said adjuvant therapeutic agents is chlorin e6.

17. The method of treating age related macular degeneration of an afflicted eye delivering adjuvant therapeutic agents through a conjunctival sac in vertebrates, to be delivered to the macula lutea, the site of the age related macular degeneration according to claim 1 further comprising the step of placing a patient on a low fat diet.

18. The method of treating age related macular degeneration of an afflicted eye delivering adjuvant therapeutic agents through a conjunctival sac in vertebrates, to be delivered to the macula lutea, the site of the age related macular degeneration according to claim 17 further comprising the step of requiring said patient to follow an aerobic exercise routine.
